(19) United States
(12) Patent Application Publication
(10) Pub. No.: US 2026/0148381 A1
(43) Pub. Date: May 28, 2026

(54) END-TO-END DIFFERENTIABLE SPECTRAL CT IMAGING

(71) Applicants: GE Precision Healthcare LLC, Waukesha, WI (US); The Board of Trustees of the Leland Stanford Junior University, Stanford, CA (US)

(72) Inventors: Sen Wang, Stanford, CA (US); Yirong Yang, Stanford, CA (US); Adam Wang, Stanford, CA (US); Grant M. Stevens, Cedarburg, WI (US)

(21) Appl. No.: 19/397,158

(22) Filed: Nov. 21, 2025

Related U.S. Application Data
(60) Provisional application No. 63/725,213, filed on Nov. 26, 2024.

(51) Int. Cl. G06T 7/00; G06T 12/30

(52) U.S. Cl. CPC G06T 7/0012; G06T 12/30; G06T 2207/20081; G06T 2207/20084; G06T 2211/408; G06T 2211/424

(57) ABSTRACT

An end-to-end differentiable image-processing pipeline for spectral Computed Tomography (CT) systems, including both Photon Counting (PCCT) and dual-energy (DECT) configurations, is provided. In the disclosed framework, spectral data acquired from a detector across two or more distinct X-ray energy spectra or energy bins are processed through a sequence of differentiable operators, including one or more differentiable preprocessing operators, a differentiable material-decomposition operator, and a differentiable image-reconstruction operator. The material-decomposition operator can be implemented using an unrolled iterative solver, an implicit-differentiation formulation, or a trained empirical proxy model. An image-domain loss between reconstructed and reference images, or other differentiable regularization criteria, is backpropagated through the pipeline to update parameters of the preprocessing operators, the material-decomposition operator, and/or a forward detector model. This enables automatic physics-informed calibration that improves quantitative image fidelity, reduces artifacts, and eliminates dependence on manual recalibration or Monte Carlo reference data.

### CROSS REFERENCE TO RELATED APPLICATIONS

This application claims priority to and the benefit of U.S. Provisional Patent Application No. 63/725,213 filed Nov. 26, 2024, the entire contents of which are incorporated by reference herein in their entirety.

### TECHNICAL FIELD

The subject disclosure relates generally to spectral Computed Tomography (CT) imaging systems, and more specifically to an end-to-end differentiable image processing pipeline for spectral imaging CT systems.

### BACKGROUND

Conventional Computed Tomography (CT) imaging systems measure the total X-ray attenuation through an object, subject, or patient, without distinguishing between photons of different energies. As a result, the reconstructed images represent a composite attenuation profile that depends on both material composition and X-ray energy spectrum, which limits the ability to differentiate between materials of similar density.

Spectral CT imaging, also referred to as multi-energy CT, extends conventional CT by acquiring energy-dependent X-ray attenuation information. This is achieved using either dual-energy (DE) CT acquisition techniques, in which projection data are obtained at two or more distinct X-ray spectra, or by employing energy-resolving photon-counting detectors that measure individual photon energies and categorize them into discrete energy bins, a technique referred to as photon-counting CT (PCCT).

To exploit the additional spectral information, spectral CT systems employ material decomposition (MD) techniques that transform the measured attenuation coefficients across different energy levels into estimates of the attenuation or density of a set of basis materials (for example, water, iodine, or calcium). In this formulation, the measured attenuation for each detector element is modeled as a weighted combination of these basis materials, each having known energy-dependent attenuation characteristics. By solving this inverse problem, spectral CT reconstructs material-specific images such as virtual monochromatic images (VMI), iodine concentration maps, and effective atomic number images that more accurately represent the underlying tissue composition. These images provide enhanced tissue characterization, improved contrast resolution, and reduction of beam-hardening artifacts compared to conventional CT, thereby supporting more accurate and quantitative diagnostic imaging.

Conventional MD algorithms typically rely on analytic or iterative reconstruction and optimization schemes, such as iterative maximum likelihood estimation. Iterative MLE for material decomposition (MD) offers asymptotically unbiased and robust (minimum variance) results, but is usually solved iteratively, making the entire process computationally expensive and time-consuming. Conversely, representative empirical methods relying on grid calibration aim to construct a direct measurement-decomposition conversion, which can be fast but may suffer from bias or noise amplification. These methods often assume ideal detector behavior and perfectly calibrated system responses, which rarely hold true in practice.

Consequently, accurate calibration of spectral CT systems is critical for achieving reliable quantitative imaging, particularly in PCCT systems that employ energy-resolving detectors, due to the need to characterize the detector's energy response across multiple bins. When calibration errors exist or system changes occur during scans, artifacts or bias in the quantitative images are observed. Calibration typically involves modeling and compensating for complex detector and system effects, including energy-dependent efficiency, charge sharing, pulse pile-up, and object scatter. In this regard, accurate and robust physical calibration and correction in spectral measurements are critical, including Photon Counting detector modeling, or other corrections such as object scatter using empirical or deep learning models. However, physical modeling and calibration of PCCT systems can be very complicated, requiring ground-truth data for calibration and training, which needs considerable human effort and time.

Conventional MD methods exacerbate these challenges because their reliance on fixed or non-differentiable algorithms makes it difficult to jointly optimize calibration parameters with image-domain objectives. When calibration errors occur, or when system characteristics drift over time, the conventional MD methods necessitate repeated and labor-intensive recalibration procedures.

Accordingly, there remains a need for improved spectral CT imaging frameworks that can automatically adapt to calibration errors and system variations without requiring extensive manual recalibration. In particular, there is a need for a differentiable imaging architecture that enables efficient end-to-end optimization of the MD and calibration processes based on quantitative image-domain objectives.

### SUMMARY

The following presents a simplified summary of the specification in order to provide a basic understanding of some aspects of the specification. This summary is not an extensive overview of the specification. It is intended to neither identify key or critical elements of the specification, nor delineate any scope of the particular implementations of the specification or any scope of the claims. Its sole purpose is to present some concepts of the specification in a simplified form as a prelude to the more detailed description that is presented later.

According to an embodiment, a computer-implemented method for generating material-specific images by a spectral CT system is described. The method includes receiving, by a system including at least one processor, spectral data including measurements acquired at two or more distinct X-ray energy spectra ranges of a spectral Computed Tomography (CT) system. The method further includes generating, by the system based on the spectral data, material decomposition (MD) values corresponding to a plurality of basis materials using a differentiable MD process configured to map the spectral data into the material decomposition values. The method further includes generating, by the system, one or more images based on the MD decomposition values.

In one implementation, the differentiable MD process includes unrolling, by the system, an iterative maximum-likelihood estimation (MLE) solver into a differentiable computational graph.

In another implementation, the differentiable MD process includes determining, by the system, analytical derivatives of the MLE solver using implicit differentiation.

In yet another implementation, the differentiable MD process includes executing, by the system, using the spectral data as input, a differentiable empirical model that generates the material decomposition values as a result of a training process used to train the differentiable empirical model. With this implementation the training process includes training the differentiable empirical model to minimize a composite loss function including: an accuracy loss term penalizing differences between training MD values output by the differentiable empirical model based on training spectral count data and reference MD values generated by an iterative MLE solver based on the training spectral count data; and a derivative loss term penalizing differences between derivatives of the differentiable empirical model and reference derivatives of the MLE solver.

According to another embodiment, an end-to-end differentiable spectral CT system is described that enables end-to-end optimization of respective components of the image processing pipeline. For example, the spectral CT system includes at least one memory storing computer-executable instructions and at least one processor configured to execute the computer-executable instructions to perform operations. These operations include receiving spectral data including measurements acquired at two or more distinct X-ray energy spectra ranges of a spectral Computed Tomography (CT) system, the spectral data having been generated by a detector of the spectral CT system. The operations further include processing the spectral data through a differentiable image processing pipeline, wherein the differentiable image formation pipeline includes a differentiable preprocessing operator that transforms the spectral data into preprocessed spectral data, a differentiable material decomposition operator that generates material decomposition values based on the preprocessed spectral data and a forward detector model of the detector, and a differentiable image reconstruction operator that generates one or more images based on the material decomposition values. The operations further include determining an image domain loss based on the one or more images, and updating one or more parameters of at least one of the differentiable preprocessing operator or the forward detector model by backpropagating gradients of the image domain loss through the differentiable image-reconstruction operator and the differentiable material-decomposition operator, resulting in a calibrated version of the differentiable image formation pipeline.

In some embodiments, elements described in connection with the disclosed systems can be embodied in different forms such as a computer-implemented method, a computer program product, or another form.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Numerous aspects, implementations, objects and advantages of the present invention will be apparent upon consideration of the following detailed description, taken in conjunction with the accompanying drawings, in which like reference characters refer to like parts throughout, and in which:

FIG. 1 illustrates an exemplary spectral CT system, in accordance with one or more embodiments described herein;

FIG. 2 illustrates another exemplary spectral CT system, in accordance with one or more embodiments described herein;

FIG. 3 illustrates a high-level block diagram of an example computing device suitable for executing image reconstruction and other computer-executable operations disclosed herein;

FIG. 4 illustrates a diagram of an example, end-to-end differentiable image processing pipeline for a spectral CT system, in accordance with one or more embodiments described herein;

FIG. 5 illustrates example results of a perturbation test used to validate the differentiable material decomposition (MD) operator configured in accordance with an implicit-differentiation method;

FIG. 6 illustrates an example method for training a neural network model to emulate the iterative MLE MD process, in accordance with one or more embodiments described herein;

FIG. 7 presents a graphical representation comparing the MD results of the proxy MD with other MD processes as applied to a physical experiment;

FIG. 8 presents comparative noise amplification results for the proxy MD and the conventional multilayer perceptron (MLP) for the physical experiment;

FIG. 9 shows the accuracy curve and noise curve along the diagonal of the test grid for the comparative MD processes evaluated in the physical experiment;

FIG. 10 illustrates the image-domain results of different MD processes in accordance with a numerical simulation experiment;

FIG. 11 illustrates the image-domain results of different MD processes in accordance with the physical experiment;

FIG. 12 illustrates an example method for calibrating parameters of the differentiable image processing pipeline based on image-domain and/or projection domain results, in accordance with one more embodiments;

FIG. 13 illustrates an example mathematical framework for gradient backpropagation in a differentiable material decomposition (MD) operator when implemented using either an unrolled iterative solver or an implicit-differentiation method, in accordance with one or more embodiments;

FIG. 14 presents image-domain results of before and after bin drift correction of the forward detector model in accordance with an experimental cross-domain calibration process;

FIG. 15 illustrates an example process for training a scatter correction model of the disclosed end-to-end differentiable image processing pipeline based on image-domain loss, in accordance with one or more embodiments;

FIG. 16 presents comparative image results of different scatter correction techniques, in accordance with an experimental evaluation;

FIG. 17 presents statistical results of the different scatter correction techniques in accordance with the experimental evaluation;

FIG. 18 presents image-domain results demonstrating the robustness of the cross-domain learning applications of the disclosed end-to-end differentiable image processing pipeline;

FIG. 19 presents a graph illustrating the robustness of a scatter correction model, trained via cross-domain learning, to mismatches between simulated and real scatter references;

FIG. 20 illustrates an example, computer-implemented method for generating material-specific images by a spectral CT system, in accordance with one or more embodiments described herein;

FIG. 21 illustrates an example, computer-implemented method for cross-domain optimization or calibration of differentiable operators of the disclosed end-to-end differentiable image processing pipeline, in accordance with one or more embodiments;

FIG. 22 is a schematic block diagram illustrating a suitable operating environment; and

FIG. 23 is a schematic block diagram of a sample-computing environment.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

The present disclosure and techniques described herein provide a spectral CT imaging framework that replaces non-differentiable material decomposition and calibration steps with differentiable counterparts and then uses quantitative image-domain objectives to automatically calibrate upstream models in a single, end-to-end optimization loop. In one aspect, a computer-implemented method receives spectral data comprising measurements acquired at two or more distinct X-ray energy spectra ranges of a spectral CT system, generates material decomposition (MD) values using a differentiable MD process, and reconstructs one or more quantitative images from those MD values.

The differentiable MD process is realized in three interchangeable ways: (i) by unrolling an iterative maximum-likelihood estimation (MLE) solver into a differentiable computational graph; (ii) by computing analytical derivatives of the iterative MLE solver via implicit differentiation; and (iii) by executing a trained differentiable empirical model, referred to herein as “proxy MD”, that emulates the iterative solver, where training uses a composite loss with an accuracy term against solver outputs and a derivative term that matches solver-derived sensitivities. These alternatives allow implementers to trade off faithfulness to the physics model, computational efficiency, and deployment constraints without changing the remainder of the pipeline.

For example, in the unrolled MLE solver embodiment, each iteration of the optimization process is explicitly represented as a differentiable operation, allowing standard automatic differentiation frameworks to propagate gradients through every step. This direct unrolling provides maximal physical fidelity to the original physics-based optimization, ensuring that the computed gradients capture the full iterative dynamics of the MLE process. The unrolled approach is particularly advantageous for smaller systems or research scenarios where interpretability and exact equivalence to the physical solver are important. However, because all intermediate iterations are stored in memory, the unrolled formulation can become computationally intensive for high-dimensional spectral CT data or large detector arrays.

In the implicit differentiation embodiment, the iterative MLE optimization is instead treated as an implicit function that satisfies a stationary-point condition. Gradients of the final solution with respect to upstream parameters are computed analytically by differentiating through this stationary condition using the implicit function theorem. This formulation achieves mathematically equivalent gradient accuracy to the unrolled solver while avoiding the need to store intermediate iterations, reducing memory consumption and computation time. Consequently, the implicit-differentiation approach offers superior computational scalability and numerical stability for large-scale Photon Counting CT (PCCT) calibration and clinical deployment while preserving the underlying physics-based model structure.

In the proxy MD embodiment, the material decomposition mapping defined by the iterative MLE solver is approximated by a trained empirical model that emulates both the quantitative outputs and local sensitivity characteristics of the MLE solution. This “proxy” model is trained using derivative-aware (Sobolev) learning, which jointly minimizes (i) the difference between the proxy's predicted MD values and those from the reference solver and (ii) the difference between their respective Jacobians. By preserving both value and derivative consistency, the proxy MD model captures the physical relationships of the MLE formulation while providing orders-of-magnitude faster inference speed during runtime. This embodiment is especially well suited for real-time applications, hardware-limited deployments, or embedded CT systems requiring rapid, deterministic execution while maintaining differentiability across the imaging pipeline.

Collectively, these three embodiments provide a continuum of implementation options: the unrolled method emphasizing transparency and physics exactness; the implicit-differentiation method emphasizing memory and computational efficiency; and the proxy-MD method emphasizing speed and deployability. Each maintains end-to-end differentiability, enabling calibration and optimization of detector models and preprocessing operators directly from quantitative image-domain objectives.

In further aspects, the system forms a differentiable image-formation pipeline that includes: (a) one or more differentiable preprocessing operators that transform raw spectral data into preprocessed spectral data; (b) a differentiable MD operator that maps the preprocessed spectral data to MD values; and (c) a differentiable reconstruction operator that generates quantitative images from the MD values. The differentiable MD operator may be implemented using any of the three methods noted above. The system computes an image-domain loss based on the quantitative images and then backpropagates gradients of that loss through the differentiable reconstruction and MD operators to upstream components (e.g., the differentiable preprocessing operators and/or a forward detector model), thereby updating model parameters and yielding a calibrated pipeline configuration. This end-to-end differentiable architecture enables the imaging chain to be optimized directly for quantitative image fidelity.

The forward detector model captures detector-specific, energy-dependent response characteristics used to generate (and interpret) the spectral counts. By positioning this model inside the gradient path, the system can automatically correct detector parameters (e.g., per-bin thresholds, gains, charge-sharing compensation) based solely on image-domain supervision, rather than manual field recalibration. The same gradient pathway also tunes differentiable preprocessing operators (e.g., denoising, normalization, bias-field and scatter correction) and reconstruction hyperparameters (e.g., regularization weights, iterative-solver step sizes) to maintain consistency between measured spectral counts and reconstructed images.

The invention thus addresses long-standing challenges of spectral CT calibration, particularly for Photon Counting systems, where accurate modeling of detector response and corrections (scatter, pulse pile-up, etc.) normally requires labor-intensive measurements or Monte-Carlo references and periodic manual recalibration. By enabling differentiable, cross-domain learning that propagates image-domain losses upstream to the physical measurement models, the system reduces or eliminates dependence on intermediate “ground-truth” references, increases robustness to reference/model mismatch, and adapts automatically to system drift. In representative demonstrations, the framework supports detector-bin-drift correction using only image-domain supervision and enables training of a scatter-correction model without direct scatter references, showing effectiveness and robustness compared with conventional empirical or direct-domain training.

Compared to conventional pipelines, the disclosed framework provides a number of technical benefits. For example, the disclosed pipeline provides: (i) a unifying, end-to-end differentiable architecture that tightly couples image quality objectives to upstream physical models; (ii) automates calibration of detector and preprocessing parameters, reducing manual effort and downtime; (iii) improves quantitative accuracy by aligning optimization with the final image domain (virtual monoenergetic images, material-density maps, etc.); (iv) offers implementation flexibility by supporting unrolled, implicit-differentiation, and learned proxy MD operators; and (v) exhibits robustness to reference mismatch and system variation, enabling reliable performance across patients, protocols, and time.

Collectively, these features provide a practical pathway to quantitative spectral CT that is calibrated by computation rather than manual operation, improving image fidelity and system stability while accelerating deployment and maintenance in clinical and research environments.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

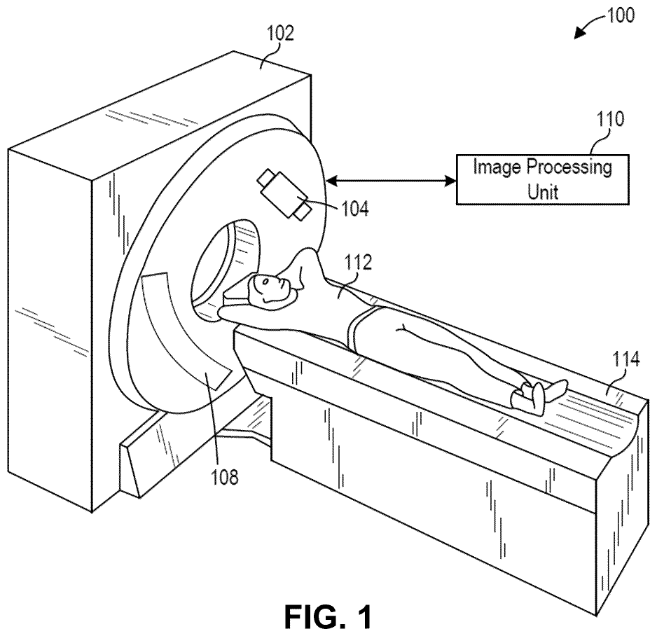

Turning now to the drawings, FIG. 1 illustrates an exemplary spectral CT system 100, in accordance with one or more embodiments. In some embodiments, spectral CT system 100 corresponds to a Photon Counting Computed Tomography (PCCT) system configured for CT imaging using a Photon Counting detector that measures individual X-ray photon energies and categorizes the detected photons into discrete energy bins. In other embodiments, spectral CT system 100 corresponds to a dual-energy (DE) CT system employing a conventional energy-integrating detector in combination with two or more distinct X-ray spectra or tube voltage settings to acquire energy-dependent projection data.

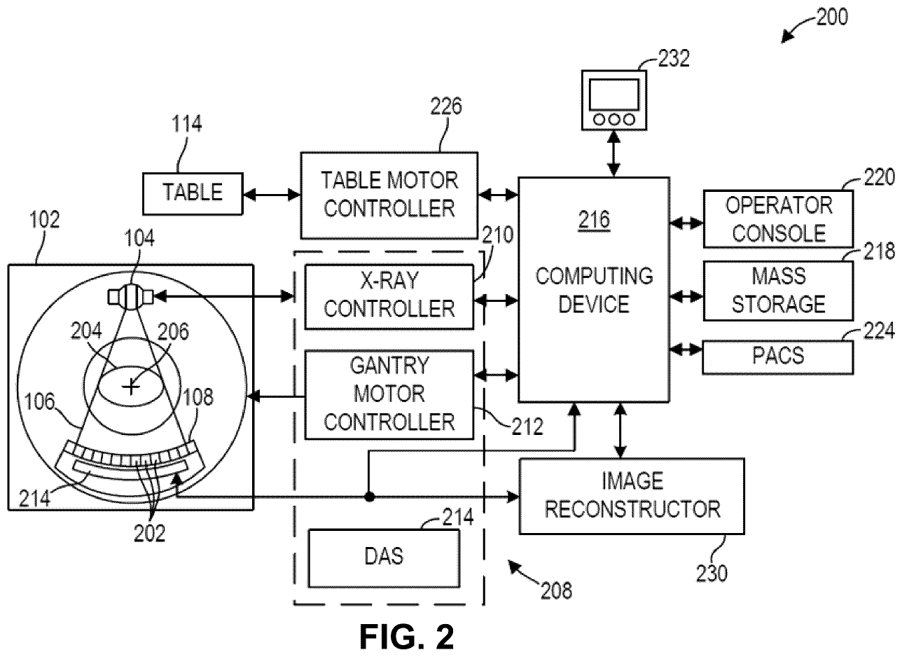

In either of these embodiments, the spectral CT system 100 is configured to image a subject 112 such as a patient, an inanimate object, one or more manufactured parts, and/or foreign objects such as dental implants, stents, and/or contrast agents present within the body. In one embodiment, the spectral CT system 100 includes a gantry 102, which in turn, may further include at least one X-ray source 104 configured to project a beam of X-ray radiation 106 (see FIG. 2) for use in imaging the subject 112 laying on a table 114. Specifically, the X-ray source 104 is configured to project the X-ray radiation beams 106 towards a detector 108 positioned on the opposite side of the gantry 102. Although FIG. 1 depicts a single X-ray source 104, in certain embodiments (e.g., DECT), multiple X-ray sources and detectors may be employed to project a plurality of X-ray radiation beams for acquiring projection data at different energy levels corresponding to the patient. In some embodiments, the X-ray source 104 may enable dual-energy gemstone spectral imaging (GSI) by rapid peak kilovoltage (kVp) switching. In the embodiments described herein, the X-ray detector employed is a Photon Counting detector which is capable of differentiating X-ray photons of different energies.

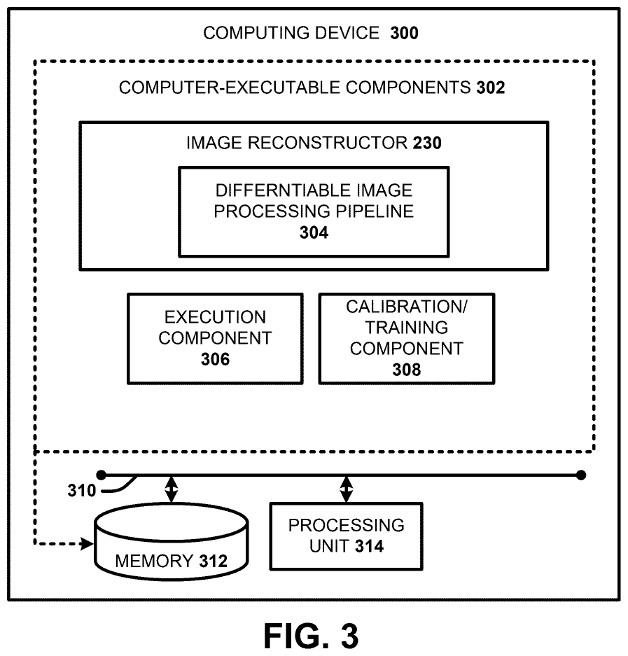

In certain embodiments, the spectral CT system 100 further includes an image processing unit 110 configured to reconstruct images of a target volume of the subject 112 using an end-to-end differentiable image processing pipeline, (described in greater detail with reference to FIG. 3 and thereafter).

In some CT imaging system configurations, an X-ray source projects a cone-shaped X-ray radiation beam which is collimated to lie within an X-Y-Z plane of a Cartesian coordinate system and generally referred to as an “imaging plane.” The X-ray radiation beam passes through an object being imaged, such as the patient or subject. The X-ray radiation beam, after being attenuated by the object, impinges upon an array of detector elements. The intensity of the attenuated X-ray radiation beam received at the detector array is dependent upon the attenuation of an x-ray radiation beam by the object. Each detector element of the array produces a separate electrical signal that is a measurement of the x-ray beam attenuation at the detector location. The attenuation measurements from all the detector elements are acquired separately to produce a transmission profile.

In some CT systems, the X-ray source and the detector array are rotated with a gantry within the imaging plane and around the object to be imaged such that an angle at which the x-ray beam intersects the object constantly changes. A group of X-ray radiation attenuation measurements, e.g., projection data, from the detector array at one gantry angle is referred to as a “view.” A “scan” of the object includes a set of views made at different gantry angles, or view angles, during one revolution of the x-ray source and detector.

FIG. 2 illustrates another exemplary spectral Computed Tomography (CT) system 200, similar to the spectral CT system 100 of FIG. 1. In some embodiments, spectral CT system 200 corresponds to a Photon Counting CT (PCCT) system. In other embodiments, spectral CT system 200 corresponds to a dual-energy CT (DECT) system. Repetitive description of like elements employed in respective embodiments is omitted for brevity.

Spectral CT system 200 is configured for imaging a subject 204 (corresponding to the subject 112 of FIG. 1). Spectral CT system 200 includes the detector 108 (see FIG. 1), which includes a plurality of detector elements 202 that together sense the X-ray beam 106 passing through the subject 204 (such as a patient or a phantom) to acquire corresponding spectral data (also referred to herein as energy-resolved projection data) comprising measurements acquired at two or more distinct X-ray energy spectra or energy-range bands. In embodiments in which spectral CT system 200 corresponds to a PCCT system, detector 108 comprises a Photon Counting detector array having a plurality of energy-resolving detector elements configured to count individual photons and assign each photon to one of a plurality of discrete energy bins, respectively corresponding to the two or more energy-range bands, according to its detected energy. Each detector element 202 outputs spectral data representing photon counts detected within each defined energy bin threshold range.

In a photon counting configuration, each detector element 202 includes a semiconductor sensor (for example, cadmium telluride (CdTe) or cadmium zinc telluride (CZT)) that directly converts incident X-ray photons into electrical charge carriers. The generated charge pulses are amplified and shaped by front-end electronics to produce a pulse whose amplitude is proportional to the photon energy. Comparator circuits or digital pulse processors compare the amplitude of each pulse against predefined energy bin thresholds to determine the energy range in which the photon belongs. Counters associated with each bin produce a histogram of photon counts versus energy, defining spectral data that preserve the energy-dependent attenuation information of the transmitted X-rays, thereby enabling quantitative spectral and material-decomposition processing.

For example, spectral data corresponding to a single projection may be represented as a vector c=[c₁, c₂, c₃, c₄], where c₁-c₄ denote photon counts measured within four distinct energy bins (e.g., 20-40 keV, 40-60 keV, 60-80 keV, and 80-100 keV). Variations in these counts reflect the energy-dependent attenuation of the X-ray spectrum caused by the subject's tissue composition and density, expressible through the underlying attenuation coefficients μ(E) along each ray path.

In embodiments in which spectral CT system 200 corresponds to a DECT system, the detector 108 comprises a dual-energy detector configuration or operates in coordination with an X-ray source capable of alternating between two or more tube-voltage settings to acquire spectral data at distinct X-ray spectra. The resulting data provide energy-resolved attenuation information equivalent, in functional terms, to the multi-bin spectral data produced by a PCCT system.

In each case, the resulting spectral data capture energy-dependent attenuation information suitable for subsequent material-decomposition and image-reconstruction processing. Accordingly, spectral CT system 200 encompasses both PCCT and DECT architectures, each configured to generate spectral data for use by the differentiable image-formation pipeline described herein.

In certain embodiments, imaging system 200 is configured to traverse different angular positions around the subject 204 to acquire spectral data from multiple view angles. Accordingly, the gantry 102 and the components mounted thereon may rotate about a center of rotation 206 to collect the spectral data at the desired energy spectra. Alternatively, in embodiments where a projection angle relative to the subject 204 varies as a function of time, the mounted components may move along a general curve rather than a circular path. As the X-ray source 104 and detector 108 rotate, detector 108 collects data of the attenuated X-ray beams, which are then subjected to differentiable preprocessing and calibration operations to condition the data to represent line integrals of attenuation coefficients of the scanned subject 204.

To exploit the spectral information, spectral CT system 200 employs a material decomposition (MD) technique that transforms the spectral data into estimates of the attenuation or density of a set of basis materials (e.g., water, iodine, calcium and/or other materials). In this formulation, the measured attenuation for each detector element is modeled as a weighted combination of these basis materials, each having known energy-dependent attenuation coefficients μₘ(E). The spectral data thereby serve as indirect measurements of the exponential attenuation of the incident spectrum, typically modeled in accordance with Equation 1 below:

$${c}_{i} \approx \int S ( E ) exp ( - {\sum }_{m} {w}_{m} {μ}_{m} ( E ) ) {R}_{i} ( E ) dE , \qquad Equation 1.$$

In Equation 1, where S(E) denotes the source spectrum, wₘ the material coefficients to be estimated, and Rᵢ(E) the detector response for the i-th energy bin. Equation 1 corresponds to a is a standard analytic expression for the Photon Counting detector forward model based on Beer-Lambert attenuation. By solving this inverse problem, spectral CT system 200 reconstructs material-specific images of the subject, such as virtual monochromatic images (VMI), iodine concentration maps, and effective atomic number (Z_eff) images, that more accurately represent the underlying tissue composition. These images provide enhanced tissue characterization, improved contrast resolution, and reduced beam-hardening artifacts compared to conventional CT, thereby supporting more accurate and quantitative diagnostic imaging.

Imaging system 200 also includes a control mechanism 208 to control movement of the components such as rotation of the gantry 102 and the operation of the X-ray source 104. In certain embodiments, the control mechanism 208 further includes an X-ray controller 210 configured to provide power and timing signals to the X-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. For example, in a spectral CT configuration, the DAS 214 converts the analog pulse signals generated by the detector elements into raw spectral data that comprising measurements acquired at two or more distinct X-ray energy spectra ranges. The data sampled and digitized by the DAS 214 is transmitted to a computer or computing device 216. It is noted that the computing device 216 may be the same or similar to image processing unit 110, in at least one example. In one example, the computing device 216 stores the data in a storage device or mass storage 218. The storage device 218, for example, may be any type of non-transitory memory and may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the X-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

Although FIG. 2 illustrates one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In one embodiment, for example, the imaging system 200 either includes, or is coupled to, a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a table 114 which may be a motorized table. Specifically, the table motor controller 226 may move the table 114 for appropriately positioning the subject 204 in the gantry 102 for acquiring projection data corresponding to the target volume of the subject 204.

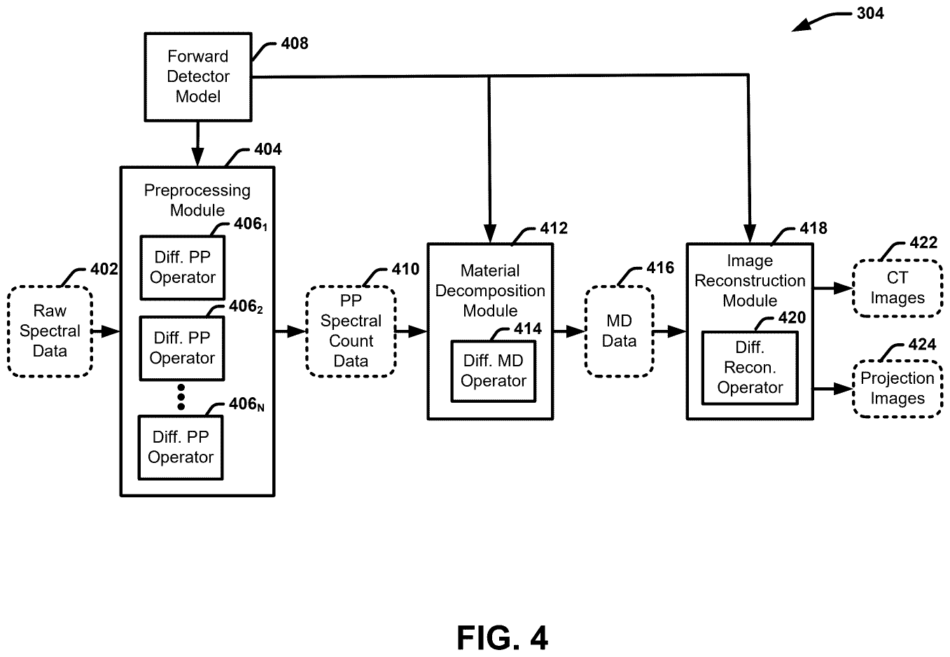

As previously noted, the DAS 214 samples and digitizes the raw spectral data acquired by the detector elements 202. Subsequently, an image reconstructor 230 uses the sampled and digitized X-ray data to perform image reconstruction. More specifically, the image reconstruction performed by the image reconstructor 230 involves processing the raw spectral data via an end-to-end differentiable image processing pipeline that includes a preprocessing step, followed by a MD step, and thereafter image reconstruction, (as described in greater detail with reference to FIG. 4 and beyond). Although FIG. 2 illustrates the image reconstructor 230 as a separate entity, in certain embodiments, the image reconstructor 230 may form part of the computing device 216. Alternatively, the image reconstructor 230 may be absent from the imaging system 200 and instead the computing device 216 may perform one or more functions of the image reconstructor 230. Moreover, the image reconstructor 230 may be located locally or remotely deployed at a separate computing device, and may be operatively connected to the imaging system 200 using a wired or wireless network. Particularly, one exemplary embodiment may use computing resources in a “cloud” network cluster for the image reconstructor 230.

In one embodiment, the image reconstructor 230 stores the images reconstructed in the storage device 218. Alternatively, the image reconstructor 230 may transmit the reconstructed images to the computing device 216 for generating useful patient information for diagnosis and evaluation. In certain embodiments, the computing device 216 may transmit the reconstructed images and/or the patient information to a display or display device 232 communicatively coupled to the computing device 216 and/or the image reconstructor 230. In some embodiments, the reconstructed images may be transmitted from the computing device 216 or the image reconstructor 230 to the storage device 218 for short-term or long-term storage.

FIG. 3 illustrates a high-level block diagram of an example computing device 300 suitable for executing image reconstruction and other computer-executable operations disclosed herein. For example, in some embodiments, computing device 300 may correspond to computing device 216. In other embodiments, computing device 300 may correspond to image processing unit 110. In other embodiments, computing device 300 may correspond to image reconstructor 230. In this regard, computing device 300 can correspond to any suitable computing device coupled to spectral imaging system 100 or imaging system 200 via any suitable wired or wireless communication framework.

In this regard, aspects of the systems, apparatuses or processes explained in this disclosure can constitute computer-executable or machine-executable component(s) or instructions embodied within machine(s), e.g., embodied in one or more computer readable mediums (or media) associated with one or more machines. In various embodiments, the computer readable mediums or media can include or correspond to a non-transitory machine-readable storage medium. Such component(s) or instructions, when executed by the one or more machines, e.g., computer(s), computing device(s), virtual machine(s), etc. can cause the machine(s) to perform the operations described with respect to the corresponding computer-executable components.

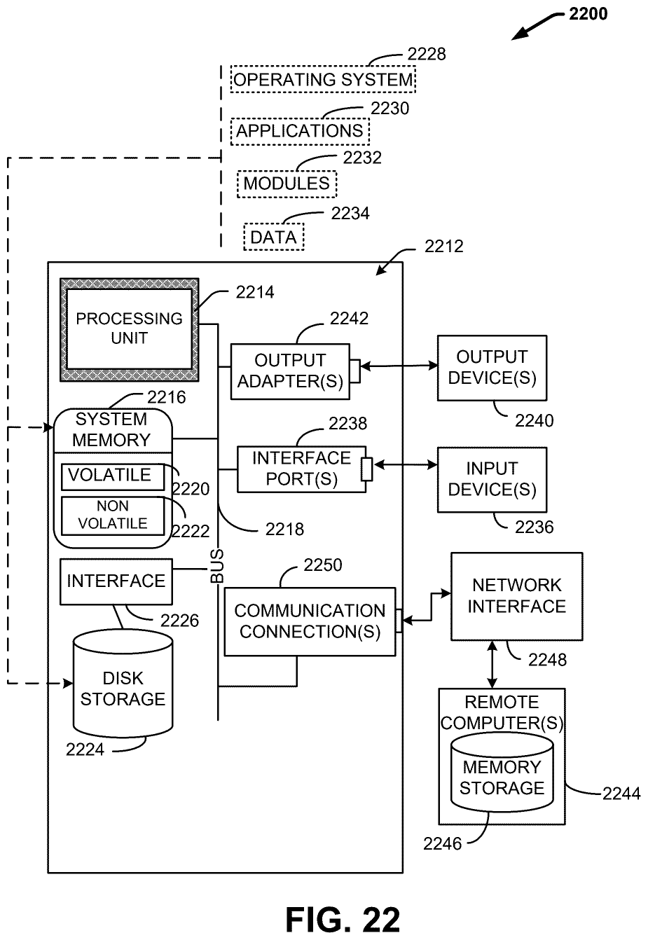

For example, computing device 300 includes a plurality of computer-executable components 302 (also referred to as machine-executable component(s), machine-executable instructions, or the like). These computer-executable components 302 include, but are not limited to, image reconstructor 230, differentiable image processing pipeline 304, execution component 306 and calibration/training component 308. It should be appreciated that these computer-executable components are stored in at least one memory 312 of the computing device 300. It should also be appreciated that the computing device 300 includes at least one processor or processing unit 314 that executes the computer-executable components stored in the at least one memory 312 to perform corresponding operations described with the respect to the computer-executable components. Computing device 300 further includes a device bus 310 that couples the memory 312 and the processing unit 314 to one another. An example of a said computing device 300, memory 312, processing unit 314, and other suitable computer or computing-based elements, can be found with reference to FIG. 22 (e.g., computing device 2212, system memory 2216 and processing unit 2214 respectively), and can be used in connection with implementing one or more the components shown and described in connection with FIG. 3, or other figures disclosed herein.

Differentiable image processing pipeline 304 (also simply referred to as pipeline 304) includes several computer-executable components that transform the raw spectral count data generated by the detector into quantitative images. Generally, the operations involved include sequentially performing preprocessing, followed by material decomposition, and then followed by image reconstruction. Additional details regarding the image processing pipeline are described with reference to FIG. 4 below.

Generally, the execution component 306 executes the imagine processing pipeline 304 on raw spectral data to generate the quantitative images. In this regard, the execution component 306 implements the forward data processing flow of the pipeline 304 to transform raw spectral data into quantitative material-decomposed or reconstructed images CT images.

In various embodiments, the pipeline 304 may be executed in a runtime mode or a calibration or training mode. In runtime mode, the execution component 306 applies the pipeline 304 to process newly acquired spectral data in a forward-only manner, generating quantitative images without performing gradient-based optimization. In calibration or training mode, the calibration/training component 308 performs optimization and learning operations that adjust parameters of one or more differentiable operators within the pipeline 304, such as the forward detector model 408, one or more differentiable preprocessing operators 406_{1-N}, and/or the differentiable material decomposition operator 414, discussed infra with reference to FIG. 4. In this mode, the calibration/training component 308 computes one or more loss functions (for example, image-domain or spectral-domain losses), propagates gradients of these losses backward through the differentiable operators of the pipeline 304, and updates associated calibration or control parameters to improve quantitative reconstruction accuracy, resulting in a calibrated version of the pipeline. The execution component 306 further executed the calibrated version of the pipeline on subsequently acquired spectral data to generate subsequent images.

Together, the execution component 306 and the calibration/training component 308 enable both operational execution of the imaging pipeline during normal scan procedures and adaptive calibration of the underlying differentiable operators to maintain or enhance performance over time.

FIG. 4 illustrates the differentiable image processing pipeline 304, in accordance with one or more embodiments described herein. Pipeline 304 corresponds to the image processing software used by image processing unit 110, image reconstructor 230, computing device 216 and/or computing device 300 to transform raw spectral data 402 into one or more CT images 422 and/or corresponding projection images 424. For instance, the raw spectral data 402 correspond to raw spectra data generated by the DAS 214 based on the analog signal data acquired via the detector 108 in association with scanning a subject (e.g., subject 112 or subject 204), such as a patient or a phantom. In other embodiments, the raw spectral data 402 may correspond to a simulated version of such acquired raw signal data as modeled using a corresponding forward detector model 408 for the detector (e.g., corresponding to Equation 1, or another forward detector model equation) for the detector 108. In either embodiments, the raw spectral data 402 comprises measurements corresponding to two or more distinct X-ray energy spectra ranges acquired via a detector of a spectral CT system (e.g., spectral CT system 100, spectral CT system 200, or the like).

In embodiments in which the spectral CT system is a DECT) system, the raw spectral data 402 corresponds to projection data acquired at two or more distinct X-ray spectra generated, for example, by alternating tube-voltage settings (e.g., 80 kVp and 140 kVp), rapid kVp-switching, or a dual-layer detector configuration. Each measurement thus represents energy-dependent attenuation of the transmitted X-rays at one of the distinct source spectra, and the combined data collectively form the raw spectral data 402 provided to the differentiable image processing pipeline 304.

In embodiments in which the spectral CT system is a PCCT system, the raw spectral data 402 correspond to spectral count data output by a Photon Counting detector array in which each detector element records photon counts across a plurality of discrete energy bin thresholds. Each bin represents a distinct energy-range response, and the multi-bin counts collectively encode the energy-dependent attenuation of the scanned subject.

Across both DECT and PCCT embodiments, the number of distinct X-ray energy spectra ranges, or discrete energy bins, may include two or more, such as three, four, five, or more, depending on detector configuration and acquisition protocol. Regardless of implementation, the raw spectral data 402 constitutes the input to the differentiable image processing pipeline 304, which performs successive preprocessing, material decomposition, and image reconstruction operations to transform the raw spectral data 402 into quantitative spectral CT images 422 and, in some cases, intermediate projection images 424 suitable for display, analysis, or further calibration.

In this regard, pipeline 304 includes a plurality of computer-executable or machine-executable component(s) (or instructions). These computer-executable include, but are not limited to, forward detector model 408, preprocessing module 404, one or more differentiable preprocessing operators 406_{1-N}, material decomposition module 412, differentiable MD operator 414, image reconstruction module 414 and differentiable reconstruction operator 420.

Pipeline 304 follows the conventional image processing chain for spectral CT systems yet uses differentiable operators for each of the processing steps, which include preprocessing, material decomposition, and image reconstruction, making the entire pipeline fully differentiable. In this regard, in a conventional spectral CT system, the image-processing chain transforms raw spectral data (e.g., corresponding to raw spectral data 402) acquired by the detector into quantitative tomographic images (e.g., corresponding to CT images 422) through a sequence of operations that typically include preprocessing, material decomposition, and image reconstruction. Following acquisition, the detector (e.g., detector 108 and/or DAS 214) generates raw spectral data 402. The raw spectral data 402 is initially subjected to preprocessing operations designed to correct and normalize the data for subsequent quantitative analysis by the downstream material decomposition and image reconstruction operations. Typical preprocessing operations include dead-time and pile-up correction to compensate for detector non-linearities and count losses, energy bin calibration to adjust for threshold drift or cross-talk between energy bins, dark-current and offset correction to remove baseline signals, scatter correction, and flat-field correction to normalize for pixel-to-pixel response variations. Additional preprocessing steps may include statistical noise filtering to reduce Poisson noise and logarithmic transformation to convert the raw spectral data into line integrals of attenuation, thereby generating calibrated spectral projection data or energy-resolved sinograms representative of the energy-dependent attenuation of the scanned object.

In various embodiments, the preprocessing module 404 performs one or more of these preprocessing operations (or alternative preprocessing operations) to transform the raw spectral data 402 into preprocessed spectral count data 410 using corresponding differentiable PP operators 406_{1-N}. The differentiable preprocessing operators 406_{1-N} may respectively correspond to one or more of the aforementioned preprocessing operations (or alternative preprocessing operations). For example, the differentiable preprocessing operators 406_{1-N} may include, but are not limited to, a differentiable spectral-response correction operator that compensates for detector-specific spectral sensitivity, a differentiable pile-up correction operator that models and corrects for pulse pile-up effects occurring at high count rates, a differentiable charge-sharing correction operator that mitigates charge-splitting among adjacent detector pixels, a differentiable scatter-correction operator that estimates and removes scattered-photon contributions, and/or a differentiable noise-normalization operator that performs variance stabilization or count-rate normalization across energy bins. In some embodiments, the preprocessing stage may further include differentiable operators for detector-gain normalization, bad-pixel suppression, dark-current subtraction, or log-linearization. Collectively, these differentiable preprocessing operators condition the raw spectral data 402 into PP spectral data 410.

In some implementations, a single differentiable preprocessing operator (e.g., diff. PP operator 406₁) may perform two or more of these preprocessing operations/tasks. In other implementations, separate preprocessing operators may respectively be configured to perform different preprocessing tasks in a chained model manner such that the output of one PP operator serves as the input to the next downstream PP operator, and so on.

Each (or in some implementation one or more) preprocessing operator 406_{1-N} is implemented as a differentiable function, such that gradients of an image-domain loss can be propagated backward through the operator during calibration. In this regard, the term “differentiable” is used herein to indicate that the corresponding operator (e.g., a model, a function, an algorithm, etc.) is differentiable. In other words, the term “differentiable” is used for each of the preprocessing operators 406_{1-N} to indicate that the respective preprocessing operators are formulated such that their input-output mappings are continuously differentiable with respect to one or more parameters or variables involved in the processing chain or pipeline. In particular, the term “differentiable” as applied to an operator, a component, the pipeline 304 and so on, is used to indicate that the corresponding mathematical or computational functions associated therewith permit analytical or numerical computation of gradients with respect to their inputs and internal parameters. As applied to the preprocessing operators, this enables the preprocessing stage to participate in an end-to-end optimization process in which gradients of an image-domain loss function can be propagated backward through the downstream material-decomposition and reconstruction operators, and further through the preprocessing operators, to update corresponding parameters of the overall pipeline 304.

Accordingly, different from conventional preprocessing modules that perform fixed or non-differentiable correction steps (e.g., lookup-table-based calibration or discrete thresholding), the differentiable preprocessing operators 406_{1-N} of the present disclosure are constructed to be differentiable components within the pipeline 304. This formulation enables joint calibration and learning of the preprocessing parameters together with other differentiable operators (e.g., differentiable material-decomposition operator 414 and differentiable image-reconstruction operator 420), thereby facilitating data-driven optimization of the full Photon Counting CT image-formation chain.

As with a conventional spectral CT image-processing chain, the preprocessed spectral data 410 are processed by a material decomposition module 412 that mathematically separates the mixed energy-dependent attenuation signals into material decomposition values (e.g., MD data 416) corresponding to a plurality of basis materials. These material decomposition values represent the relative contributions of the basis materials (for example, water, iodine, calcium, and/or other materials) to the measured attenuation across the acquired energy spectra or energy bins. In some embodiments, the material decomposition operation is performed in the projection domain, wherein the energy-dependent spectral data are decomposed before image reconstruction. In other embodiments, the operation is performed in the image domain, wherein separate energy-specific or energy bin images are first reconstructed and subsequently decomposed on a voxel-wise basis. The same mathematical formalism may thus be applied to both PCCT and DECT configurations, differing primarily in how the underlying energy spectra are acquired and represented.

Conventional MD techniques generally employ analytical or iterative optimization methods, such as maximum-likelihood estimation (MLE), to solve the nonlinear relationship between the measured spectral count data and the attenuation coefficients of the basis materials. The output of this stage comprises material decomposition values (e.g., MD data 416) that collectively define a set of basis-material sinograms or images representing the spatial distribution of each corresponding material component. As described in greater detail below, conventional MD techniques such as MLE are not differentiable and thus prevent the realization of a fully differentiable image processing pipeline, such as pipeline 304. Different from conventional MD techniques, pipeline 304 replaces the conventional non-differentiable MD operator with a differentiable MD operator 414 that generates the MD data 416 using a differentiable MD process.

As with the conventional PCCT image processing chain, the material-decomposed or energy-resolved data (e.g., MD data 416) are then passed to the image reconstruction module 318, which converts the projection data into CT images 422 and in some implementations, corresponding projection images 424. The CT images 422 may include any reconstructed tomographic images derived from the material decomposition values, such as basis-material images (for example, water-, iodine-, or calcium-basis images), virtual-monochromatic images (VMIs) synthesized at selected photon energies, effective atomic-number (Zeff) maps, and/or electron-density images.

The projection images 424, by contrast, may include forward-projected or reprojected spectral data generated from the reconstructed CT images 422 or intermediate basis-material maps. Such projection images can represent estimated line integrals, spectral projections, or sinograms at selected energy levels or basis-material combinations. In some embodiments, these projection images are used for calibration, consistency checking, or loss-function computation during end-to-end training/calibration of pipeline 304, or system verification. Thus, CT images 422 provide reconstructed volumetric representations in the image domain, while projection images 424 correspond to synthesized or residual data in the projection domain that preserve the physical acquisition geometry. Both are outputs of the differentiable reconstruction module 318 and may be utilized individually or jointly for visualization, quantitative analysis, or feedback calibration within the differentiable image-formation pipeline.

Conventional techniques used to perform reconstruction of the MD data 416 include using reconstruction algorithms such as filtered back-projection (FBP) or iterative reconstruction (IR) methods. In projection-domain MD, each material-specific sinogram is reconstructed independently to form a corresponding image, whereas in image-domain MD, decomposition is applied to reconstructed energy bin images to yield the final material maps.

Different from conventional spectral CT imaging processing pipelines, pipeline 304 also uses a differentiable reconstruction operator 420 for the reconstruction phase. In this regard, the differentiable reconstruction operator 420 is configured to generate the CT images 422 and/or the projection images 424 from the MD data 416 using a reconstruction function that is continuously differentiable with respect to its input data and internal parameters. The differentiable reconstruction operator 420 may be implemented using one or more analytical, iterative, or learned reconstruction models that are reformulated or approximated in a differentiable manner to enable gradient-based optimization. For example, in some embodiments, the differentiable reconstruction operator 420 corresponds to a differentiable version of a conventional filtered back-projection (FBP) algorithm, wherein both the filtering and back-projection steps are implemented using convolutional or matrix-based operations that are differentiable with respect to the input sinogram data and system geometry parameters. In other embodiments, the differentiable reconstruction operator 420 may correspond to an iterative reconstruction model such as an algebraic reconstruction technique (ART), simultaneous algebraic reconstruction technique (SART), or maximum-likelihood expectation-maximization (MLEM) formulation, implemented as an unrolled sequence of differentiable update steps that enable gradient propagation through the iterative process. In still further embodiments, the differentiable reconstruction operator 420 may comprise a neural network model trained to approximate or enhance conventional reconstruction functions, such as a convolutional neural network or a learned primal-dual reconstruction network configured to perform data consistency enforcement and artifact suppression while maintaining differentiability throughout the computational graph.

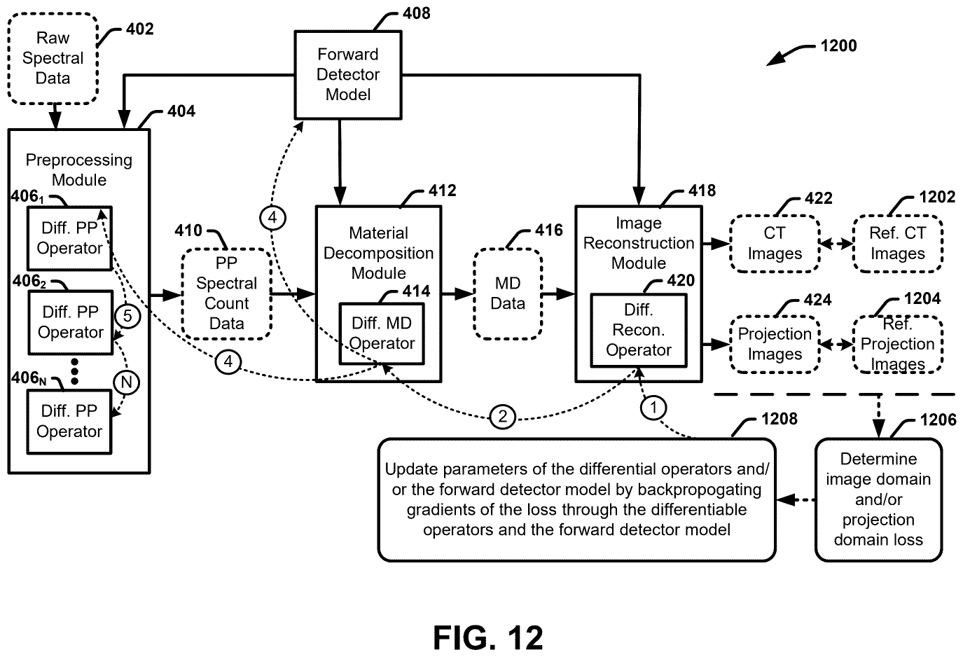

In some embodiments, the differentiable reconstruction operator 420 includes one or more trainable parameters, such as regularization weights, filter coefficients, or kernel parameters, which may be updated during end-to-end optimization of the image processing pipeline based on gradients propagated from an image-domain loss function, as described in greater detail with reference to FIG. 12. The differentiable formulation of the reconstruction operator thus enables joint calibration and learning across the full imaging chain, allowing the reconstruction process to adapt to characteristics of the forward detector model 408, the one or more differentiable preprocessing operators 406_{1-N}, and the differentiable MD operator 414. In contrast to conventional fixed reconstruction algorithms that are not differentiable with respect to upstream processing parameters, the differentiable reconstruction operator of the present disclosure provides a continuous and trainable mapping from spectral projection data to quantitative CT images, enabling data-driven optimization of the Photon Counting CT image-formation pipeline as a unified differentiable system.

Pipeline 304 also includes a forward detector model 408, which represents a computational model that characterizes the spectral-response behavior of the detector (e.g., detector 108) of the spectral CT system. The forward detector model 408 provides a differentiable mapping from underlying material-dependent attenuation or projection data to the spectral data measured by the detector, thereby capturing how the physical detector converts incident X-ray photons of varying energies into energy-resolved measurements. As described above with respect to Equation 1, this mapping relates the energy-dependent attenuation of the X-ray spectrum to the expected detector output, whether expressed as photon counts across energy bins in a PCCT configuration or as intensity measurements at multiple source spectra in a DECT configuration.

In various embodiments, the forward detector model 408 simulates or parameterizes one or more aspects of detector physics, including photon interactions, charge sharing, pulse pile-up, dead-time effects, spectral cross-talk, energy bin thresholding, spectral-response broadening, and quantum efficiency. The forward detector model 408 is parameterized by one or more detector parameters that characterize the detector's spectral-response properties, such as energy bin thresholds, per-pixel gain coefficients, per-channel offset coefficients, detector dead-time coefficients, pile-up coefficients, charge-sharing correction factors, cross-talk correction factors, bin spectral-response functions, quantum-efficiency parameters, detection-probability parameters, spatial-drift correction terms, temporal-drift correction terms, noise-correlation parameters, and dark-count parameters. These parameters may vary across detector elements, energy ranges, or acquisition conditions.

The forward detector model 408 is implemented as a parameterized differentiable operator that approximates or generalizes the physical relationship described by Equation 1. Its mathematical operations, including spectral-response weighting, energy integration, and statistical noise modeling, are continuously differentiable with respect to both its inputs and internal parameters. This differentiability enables the model to participate in gradient-based optimization of the overall image-formation pipeline while maintaining physical interpretability.

During normal imaging operation, the (optionally calibrated) forward detector model 408 is used within pipeline 304 to interpret and process the spectral data produced by the detector. In some embodiments, the forward detector model 408 may be invoked by one or more differentiable preprocessing operators 406_{1-N} to correct raw spectral count data, guide normalization or threshold adjustment. Additionally, or alternatively, the forward detector model 408 may be used by the differentiable MD operator 414 in association with performing the differentiable MD process. In this role, the forward detector model 408 provides the physical mapping that relates material-basis coefficients or attenuation values to the corresponding spectral measurements, such that the differentiable MD operator 414 can estimate basis-material contributions consistent with the modeled detector response. For example, the forward detector model 408 may provide spectral-weighting factors, response functions, or energy-integration terms/parameters that are used by the MD operator to accurately transform spectral data into material-decomposition values. The forward detector model 408 may further be used by the differentiable reconstruction operator 420 to account for detector spectral-response effects in generating CT images 422 and/or projection images 424.

In some embodiments, the forward detector model 408 can also operate in a simulation mode to generate synthetic spectral data that replicates the characteristics of the physical detector for training, validation, or quality-assurance purposes. By integrating the forward detector model 408 within the differentiable image-formation pipeline 304, the imaging system (e.g., systems 100, 200, or the like) maintains consistent, physics-based modeling of detector response across both calibration and runtime modes, thereby enhancing the quantitative accuracy and robustness of reconstructed CT images 422.

In this regard, spectral CT detectors, including both Photon Counting detectors and dual-energy detector configurations, provide energy-dependent measurements of transmitted X-ray photons, thereby enabling material decomposition (MD) that separates attenuation into constituent basis materials. The measured spectral data, however, are affected by various non-ideal detector and acquisition effects, including finite spectral response, cross-talk between energy channels, and statistical noise. In conventional spectral CT systems, such effects are compensated by incorporating a forward detector model 408 into the MD process and solving a maximum-likelihood-estimation (MLE) problem that iteratively matches measured spectral data to predicted spectral responses, or by applying empirical calibration methods such as material-grid or look-up-table approaches. Iterative MLE methods generally achieve asymptotically optimal accuracy and noise performance (approaching the Cramér-Rao lower bound) but are computationally expensive due to their iterative nature. Conversely, purely empirical or calibration-based methods are computationally efficient but often yield sub-optimal noise performance and limited generalizability.

In accordance with the differentiable image-formation pipeline 304, the differentiable MD operator 414 is formulated as a differentiable version of the MLE-based MD process. In this formulation, the differentiable MD operator 414 optimizes a likelihood function representing the agreement between measured spectral data and predicted measurements generated by the forward detector model 408, optionally subject to one or more regularization constraints. Because conventional MLE solvers are typically non-differentiable with respect to upstream parameters, such as detector thresholds, spectral-response functions, and other parameters of the forward detector model 408 or preprocessing transforms, they cannot directly participate in end-to-end optimization of the image-formation pipeline. To overcome this limitation, the differentiable MD operator 414 is configured as a differentiable formulation of the MLE solver, enabling gradients of an image-domain loss to be backpropagated through the MD operation and upstream through other differentiable components of the pipeline.

In certain embodiments, the differentiable MD operator 414 is implemented using an iterative maximum-likelihood estimation (MLE) solver that has been rendered differentiable by unrolling its optimization steps into a computational graph. In this embodiment, the differentiable MD operator 414 performs a differentiable MD process that comprises unrolling an iterative MLE solver into a finite sequence of differentiable layers. For example, a Newton-based or gradient-descent solver may be represented as a sequence of iterative updates expressed as Equation 2:

$${A}_{n + 1} = {A}_{n} + φ ( {A}_{n} ; {γ}_{c} , {θ}_{λ} ) \qquad Equation 2.$$

In Equation 2, where Aₙ denotes an intermediate material decomposition estimate at iteration n, and φ(·) represents a differentiable update function derived from the MLE optimization criterion. The parameter set γ_{c} represents calibration or control parameters associated with the material decomposition process, such as step-size coefficients, regularization weights, or convergence criteria that govern the iterative updates, while θ_{λ} denotes detector-model parameters, such as energy thresholds, gain, and spectral-response coefficients. The final estimate A*may be defined as the minimizer of a material decomposition loss function, represented for example by Equation 3:

$${A}^{*} ∈ arg {min}_{A} {L}_{M D} ( A ; {γ}_{c} , {θ}_{λ} ) , \qquad Equation 3.$$

In Equation 3, L_{MD} denotes a material-decomposition objective function parameterized by calibration parameters γ_{c} and detector model parameters θ_{λ}.

In accordance with the unrolled method, when executing the pipeline 304 in calibration or training mode (e.g., via execution component 306 and calibration/training component 308), the differentiable MD operator 414 receives the PP spectral count data 410 and applies the iterative update rule of Equation 2 to produce intermediate estimates Anand a final estimate A*, corresponding to MD data 416. The image reconstruction module 418 then transforms the MD data 416 into one or more CT images 422 using differentiable reconstruction operator 420. The calibration/training component 308 then computes an image-domain loss (for example, between the reconstructed CT images 422 and corresponding reference images), and backpropagates gradients of that loss through the unrolled iterations of the MD operator 414 and through upstream components such as the forward detector model 408 and/or one or more of the differentiable PP operators 406_{1-N}. This operation enables end-to-end optimization of the parameters γ_{c} of the differentiable MD operator 414, the parameters θ_{λ} of the forward detector model 408, and parameters of the differentiable PP operators 406_{1-N} thereby calibrating the pipeline 304 to improve quantitative accuracy.

During a runtime mode, the same differentiable MD operator 414 is executed in a forward-only configuration using the calibrated parameters γ_{c}* and θ_{λ}* obtained during calibration. For example, during a runtime mode the pipeline 304 receives new spectral count data from a patient scan, and the differentiable MD operator 414 applies the iterative update rule of Equation (2) without computing or storing gradients, and outputs final material decomposition values A* for subsequent reconstruction by the differentiable reconstruction operator 420. Because backpropagation and gradient storage are disabled in runtime mode, computational and memory demands are greatly reduced, enabling efficient deployment of the trained differentiable pipeline for clinical imaging.

Accordingly, the unrolled differentiable solver functions both as a calibration mechanism, enabling gradient-based optimization during training, and as an operational inference module that deterministically computes material-decomposition values using optimized parameters.

In another embodiment, the differentiable MLE solver is implemented using implicit differentiation. In this embodiment, the differentiable MD operator 414 performs a differentiable material decomposition process in which the iterative MLE optimization is treated as an inner optimization problem whose solution A*satisfies a stationary condition of the form of Equation 4.

$$\frac{\partial F ( {A}^{*} , {γ}_{c} , {θ}_{λ} )}{\partial A} = 0 \qquad Equation 4.$$

In Equation 4, F denotes the MLE objective function, A represents the material-decomposition variables, γ_{c} denotes calibration parameters, and θ_{λ} denotes parameters of the forward detector model 408, such as energy thresholds or spectral response coefficients. In this formulation, gradients of an outer loss function Lₒᵤₜₑᵣ(A*(γ_{c}, θλ)), for example, an image-domain reconstruction loss, are computed analytically by differentiating through the stationary condition using the implicit function theorem represented in Equation 5.

$$\frac{d {L}_{outer}}{d {θ}_{λ}} = - {( \frac{{\partial }^{2} F}{\partial {A}^{2}} )}^{- 1} \frac{{\partial }^{2} F}{\partial A \partial {θ}_{λ}} \frac{\partial {L}_{outer}}{\partial A} . \qquad Equation 5.$$

This relationship enables exact gradient computation with respect to upstream parameters without unrolling the iterative solver or storing intermediate variables. During training or calibration mode, the calibration/training component 308 invokes the differentiable MD operator 414 to compute analytical derivatives of the stationary solution A*with respect to γ_{c} and θ_{λ}. The image domain loss is then backpropagated through these analytical gradients to update the parameters of the differentiable MD operator 414, the forward detector model 408, and one or more of the differentiable preprocessing operators 406_{1-N}. Because this method avoids storing the entire sequence of MLE iterations, GPU memory usage scales sublinearly with the number of solver steps, making it particularly efficient for Photon Counting CT systems involving thousands of detector elements or high-resolution projection data.

During runtime mode, the same calibrated differentiable MD operator 414 executes the MLE optimization forward-only to compute the stationary solution A* (γ_{c}*, θ_{λ}*) using the optimized parameters obtained during training/calibration. No gradient computation or storage is performed in runtime mode, resulting in a deterministic, memory-efficient inference process that yields quantitative material decomposition values suitable for reconstruction by the differentiable reconstruction operator 420. Thus, the implicit differentiation based MLE solver achieves mathematically equivalent gradient accuracy to the unrolled solver while providing substantial improvements in computational efficiency and scalability for large-scale PCCT calibration and clinical deployment.

To validate the differentiable MD operator 414 as configured in accordance with the implicit-differentiation method, a perturbation test was performed to confirm that the analytical gradients produced by implicit differentiation accurately represent the local sensitivities of the iterative MLE MD solver. In this test, small perturbations were applied to detector threshold parameters around a reference data point corresponding to 10 cm of water and 1 cm of calcium, and the resulting changes in material decomposition (MD) outputs were compared with the predicted changes obtained from the analytically computed gradients.

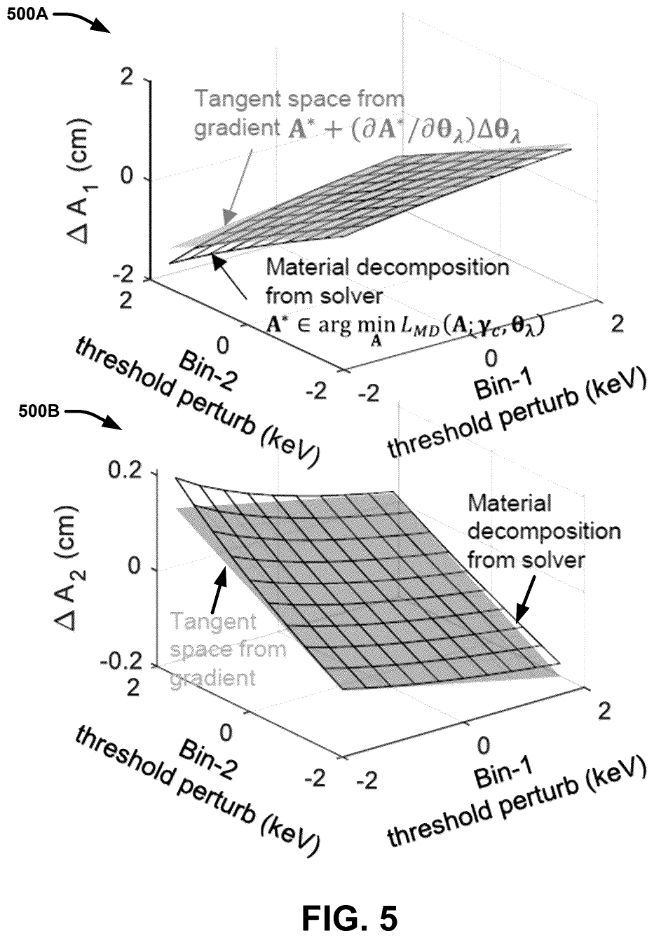

FIG. 5 illustrates example results of the perturbation test. The upper plot 500A shows variations (ΔA₁) in the estimated amount of a first basis material as a function of threshold perturbations applied to two energy bins, while the lower plot 500B shows corresponding variations (ΔA₂) for a second basis material. In each plot, the material decomposition from the solver surfaces represent the MD results obtained by directly running the iterative MLE solver under perturbed detector thresholds, whereas the tangent space surfaces from the gradient planes represent the analytical first-order predictions computed from the implicit differentiation gradients, (∂A*/∂θλ). As seen in FIG. 5, the tangent space planes closely align with the solver-based MD surfaces across the range of threshold perturbations, confirming that the analytically derived gradients faithfully capture the true parameter sensitivities of the iterative MLE solver. This validation demonstrates that the implicit differentiation-based MD operator accurately models the local response of the MLE solver and can therefore be reliably used in downstream calibration and optimization tasks.

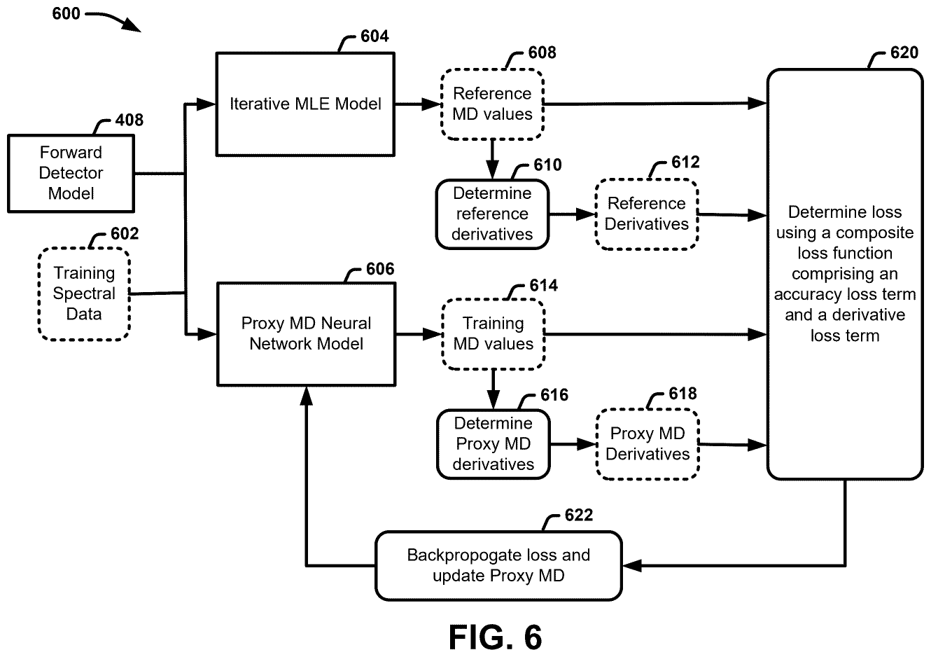

While both unrolled and implicit differentiation techniques permit end-to-end gradient propagation through iterative MLE solvers, their computational burden can remain significant for high-dimensional PCCT data. Accordingly, in further embodiments, the implicit material decomposition mapping defined by the differentiable MLE solver is itself approximated by a differentiable empirical model, referred to herein as proxy MD model 606 (or simply “proxy MD”), shown in FIG. 6. With these embodiments, the differentiable MD operator 420 is or corresponds to the proxy MD model 606 (or simply proxy MD).

The proxy MD model 606 implements an empirical differentiable mapping that directly transforms the PP spectral data 410 into material decomposition values (e.g., MD data 416) without explicitly executing the iterative MLE solver at runtime. In various embodiments, the empirical model may take different forms, including but not limited to a neural network model (for example, a multilayer perceptron (MLP), convolutional neural network (CNN), or transformer-based architecture), a parametric regression model, a kernel or Gaussian-process model, a mixture-of-experts ensemble, a differentiable lookup table, or another trainable function approximator that is continuously differentiable with respect to its inputs and parameters. Each such empirical model is parameterized by learnable weights or coefficients and trained to emulate the outputs and sensitivities of the reference MLE solver using a composite loss function that includes an accuracy term and a derivative-consistency term, as further described below.

By employing such an empirical model, the differentiable MD operator 414 (proxy MD) achieves the accuracy of the MLE solver while dramatically reducing computational complexity and enabling real-time or near-real-time execution during pipeline inference. In this regard, the duration of time used by the system (e.g., spectral CT system 100, spectral CT system 200, or the like) for executing the proxy MD model 606 to generate the material decomposition values is significantly lower than a corresponding duration of time required by the system to execute the iterative MLE solver to produce corresponding material decomposition values based on the PP spectral data 410. For example, in some embodiments, the proxy MD model 606 executes one to three orders of magnitude faster than the iterative MLE solver, reducing computation time from several seconds or minutes per volume to tens of milliseconds per volume, thereby enabling near-real-time spectral image reconstruction and interactive calibration within the differentiable image processing pipeline 304.

The proxy MD model 606 thus serves as a learned, differentiable surrogate for the iterative MLE process, maintaining physical consistency with the forward detector model 408 and supporting gradient propagation throughout the entire image processing pipeline 304. In this embodiment, rather than explicitly solving the stationary condition of the MLE formulation for each input, the calibration/training component 308 trains the proxy MD model 606 to emulate the implicit function A*(γ_{c}, θ_{λ}) produced by the iterative MLE solver. The proxy MD is trained using derivative-aware (Sobolev) learning, wherein the network is optimized not only to match the material decomposition outputs generated by the implicit solver but also to reproduce the corresponding analytical gradients ∂A*/∂(γ_{c}, θ_{λ}). This dual-term supervision enables the neural proxy to preserve the physical consistency and noise characteristics of the differentiable MLE solution while providing substantially accelerated inferencing speed. As described in further detail below, deployment of the proxy MD within the pipeline 304 enables real-time execution of the material decomposition process with speedups exceeding two orders of magnitude relative to the iterative MLE solver while maintaining quantitative accuracy and end-to-end differentiability for calibration and detector model correction tasks.

FIG. 6 illustrates an example training process 600 for training the proxy MD model 606 to emulate the iterative MLE MD process, in accordance with one or more embodiments described herein. In training process 600, both accuracy and derivative (Jacobian) constraints are incorporated to ensure that the proxy MD neural network model 606 reproduces not only the quantitative outputs of the iterative MLE model 604 (corresponding to an iterative MLE solver) but also its local sensitivity characteristics. As shown, the training spectral data 602 and the forward detector model 408 serve as the common inputs to both the iterative MLE model 604 and the Proxy MD model 606.

The training spectral data 602 may correspond to the raw spectral data 402 or the preprocessed form thereof (PP spectral data 410). In one embodiment, the training spectral count data 602 corresponds to energy-resolved photon counts acquired across multiple detector energy bins and projection angles, such as a counts sinogram obtained from a calibration or phantom scan (or a high-resolution diagnostic scan). In various embodiments, the training spectral data 602 may include measured data acquired during calibration or diagnostic scans, such as data obtained from a reference phantom having known basis materials with predetermined attenuation coefficients, or from patient imaging data collected under clinical protocols. In other embodiments, the training spectral data 602 may comprise simulated spectral data generated using the forward detector model 408. In each case, the training spectral data 602 serves as representative input examples for training the proxy MD model to emulate the material-decomposition mapping defined by the reference MLE solver or corresponding ground-truth material values.

It should be appreciated that the training process 600 involves a plurality of iterations, wherein at each iteration, a different version of the training spectral data 602 corresponding to a different CT scan (e.g., a calibration scan, a phantom scan, or a diagnostic scan) is respectively processed as input to the respective models (e.g., the iterative MLE model 604 and the proxy MD model 606). The forward detector model 408 is also used as input to both the MLE model 604 and the proxy MD model 606. As noted above, the forward detector model 408 encodes detector specific parameters (e.g., threshold settings, gain factors, and spectral-response coefficients) that are used to transform the input counts into the physical quantities required for material decomposition. Accordingly, at each training iteration, the same spectral data 602 and the forward detector model 408 are separately processed by both the iterative MLE model 604 and the Proxy MD neural network 606 to produce respective MD outputs.

The iterative MLE model 604 implements a physics-based optimization procedure, such as a Newton or gradient-descent solver, that minimizes an MLE objective function to estimate basis-material densities or attenuation coefficients. The final solution produced by the iterative MLE model 604 serves as the reference MD values 608, which are regarded as the ground-truth targets for training. The proxy MD model 606, when applied to the same inputs, generates corresponding training MD values 614, which represent the network's predicted material decomposition results.

To enable derivative-aware training, both models are also subjected to differentiation operations that quantify how their outputs change with respect to variations in the training spectral data 602 (e.g., noise variations or perturbations). For example, during training process 600, at 610 the calibration/training component 308 determines the reference derivatives 612 of the iterative MLE model 604, and at 616 the calibration/training component determines the proxy MD derivatives 618 of the empirical proxy MD model 606.

In certain embodiments, the reference derivatives 612 are computed by evaluating the Jacobian of the iterative MLE model 604 using implicit differentiation, in which analytical gradients of the stationary conditions of the MLE objective are evaluated to obtain sensitivities. In other embodiments, the reference derivatives 612 may be determined using an unrolled iterative approach, in which the MLE optimization steps are explicitly unfolded into a differentiable computational graph and differentiated by backpropagation through the unrolled iterations. Either approach provides reference derivative information representative of the noise and sensitivity behavior of the iterative MLE process, which the empirical proxy MD model 606 is trained to emulate through its derivative loss term. The corresponding proxy MD derivatives 618 are obtained by automatic differentiation of the proxy MD neural network 606 with respect to its inputs. These derivatives respectively represent the local sensitivity of the MD outputs to variations in the input spectral counts, including variations arising from photon noise or detector non-idealities. In this regard, the proxy MD derivatives 618 and the reference derivatives 612 respectively represent how the training MD values 614 and the reference MD values 608 change in response to variations in the training spectral data 602.

At step 620, the calibration/training component 308 performs a loss evaluation using a composite loss function that combines two complementary terms, including an accuracy loss term and a derivative loss term. The accuracy loss term penalizes (first) differences between the training MD values 614 and the reference MD values 608, thereby enforcing quantitative agreement between the network's outputs and the MLE ground truth. The derivative loss term penalizes (second) differences between the proxy MD derivatives 618 and the reference derivatives 612, thereby constraining the network to reproduce the local response characteristics (e.g., noise behavior and gradient structure) of the MLE solver. In other words, the derivative loss term constrains the proxy MD model 606 to replicate noise response characteristics of the iterative MLE model 604.

In this regard, at 620, the calibration/training component 308 determines a composite loss measure that combines both an accuracy loss value and a derivative loss value. For example, in some embodiments, the composite loss Lₜₒₜₐₗ may be expressed as Equation 6.

$${L}_{total} = α {‖ {A}_{Proxy} - {A}_{MLE} ‖}^{2} + β {‖ \frac{\partial {A}_{Proxy}}{\partial I} - \frac{\partial {A}_{MLE}}{\partial I} ‖}^{2} , \qquad Equation 6.$$

In Equation 6, A_{proxy} and A_{MLE} respectively denote the training and reference MD values, I denotes the input spectral count data, and α, β are weighting coefficients that balance accuracy and derivative fidelity.

As shown at 622, the composite loss is backpropagated through the proxy MD model 606 to update its learnable parameters using stochastic-gradient or adaptive-moment optimization. Through this process, the proxy MD model 606 learns an explicit approximation of the implicit MLE function and its Jacobian, such that the network not only reproduces the quantitative MD outputs but also mimics the MLE model's noise response and sensitivity profile.

During subsequent inference (runtime) operations, the trained version of the proxy MD model 606 replaces the iterative MLE solver as the differentiable MD operator 414, enabling rapid forward evaluation of material decomposition values without explicit optimization, thereby achieving runtime accelerations exceeding two orders of magnitude (or more) while maintaining the physical accuracy and stability of the differentiable MLE based process. In this regard, during runtime or inference mode, the trained version of the proxy MD model 606 operates as an explicit, forward-only substitute for the iterative MLE model 606 within the differentiable image processing pipeline 304. In this configuration, the proxy MD model 606 receives new spectral count data (or more particularly PP spectral count data 410), such as projection-domain measurements acquired from a patient scan, and the corresponding forward detector model 408 parameters (e.g., calibrated energy thresholds and gain factors θ_\λ*). Using these inputs, the proxy MD model 606 directly outputs quantitative material decomposition values A* (e.g., corresponding to MD data 416), bypassing iterative optimization altogether. Because gradient computation and intermediate state storage are disabled in this mode, the proxy MD model 606 executes with minimal memory and latency, typically yielding real-time throughput suitable for clinical imaging applications.

Despite its compact runtime form, the proxy MD model 606 remains fully differentiable with respect to upstream parameters of the forward detector model 408 and the one or more differentiable preprocessing operators 406_{1-N}, thereby preserving end-to-end differentiability of the pipeline 304 for any subsequent fine-tuning or cross-domain optimization. Accordingly, the proxy MD embodiment provides a unified framework in which the physics-based differentiability of the MLE formulation is retained while achieving more than two orders of magnitude of processing acceleration in the material-decomposition stage.

In this regard, training process 600 is based on a theoretical analysis of accuracy and noise performance of iterative MLE MD from a perspective of implicit functional mapping between measurement and MD results. The function value of the implicit function means that the decomposition accuracy and the first-order derivatives (Jacobian) provide information about the decomposition noise. The inventors of the disclosed subject matter have realized that such a mapping can be learned using an empirical approximator (e.g., a polynomial, a rational polynomial, a neural network, a regression function, or other trainable function approximators) by incorporating both an accuracy loss term and a derivative loss term (to account for noise responses) in the training. This realization aligns with the concept of Sobolev training. The implicit function theorem also explains why conventional empirical models often perform suboptimally in their noise behavior since they usually only incorporate the accuracy term in their fitting and training.

In the diagnostic energy range, material attenuation coefficients can be linearly represented by basis functions or basis materials (e.g., water and calcium), as shown in Equation 7 below.

$$μ ( E ) = {\sum }_{i = 1}^{M} {α}_{i} {μ}_{i} ( E ) \qquad Equation 7.$$

In Equation 7, μ(E) are the material attenuation coefficients, μᵢ(E) is the mass attenuation of the i-th basis material as a function of energy (E), αᵢ is the decomposition coefficient, and M denotes the number of basis materials.

Material decomposition estimates the line integrals (thickness) A ∈ [symbol] from spectral measurements γ∈ [symbol] from detectors based on some objective function L(·). Assuming that the measure counts follow a Poison noise model, the corresponding objective function may be modeled as the log-likelihood function in Equation 8 below:

$${A}^{*} ϵ arg \begin{matrix}min \\ A\end{matrix} L ( A ; γ ) , L ( A ) = {\sum }_{n} {f}_{n} ( A ; {θ}_{λ} ) - {γ}_{n} log ( {f}_{n} ( A ; {θ}_{λ} ) ) \qquad Equation 8.$$

In Equation 8, fₙ(·) is the n-th energy bin in the forward detector model 408, θ_{λ} are the parameters in the forward detector model 408, and γₙ are the measurements in the n-the energy bin. The estimated line integrals A* (e.g., the MD values) can then be reconstructed into CT images 422 by the differentiable reconstruction operator 420 (e.g., basis images and VMIs) for downstream tasks.

In various embodiments, the iterative MLE model 604 comprises a conventional iterative MLE solver configured to generate the reference MD values 608 by iteratively solving Equation 8, such as the Nelder-Mead or Newton Raphson solver. These methods are commonly used to solve equation 8 by finding the stationary point using Equation 9 below.

$${H ( {A}^{*} , γ ) = \frac{\partial L ( A ; γ )}{\partial A} \vert}_{A = {A}^{*}} = 0 \qquad Equation 9.$$

The value of A*is implicitly defined by γ and H(·). From the implicit function theorem, the functional mapping between A* and γ exists when ∂H(A*; γ)/∂A*is invertible (nonzero). Then, the material decomposition is reformulated as an implicit function h(γ) in Equation 10, where the exact analytical expression of h is usually unavailable.

$${A}^{*} = h ( γ ) when \partial H ( {A}^{*} ; γ ) / \partial {A}^{*} is non - singular \qquad Equation 10.$$

The noise properties can further be obtained through Jacobian analysis of Equation 10, which can be realized by unrolling the iterative solver or using the implicit differentiation theorem, as shown in Equation 11.

$$h ( γ ) \approx h ( \overline{γ} ) + ∇ h ( \overline{γ} ) ( γ - \overline{γ} ) , where ∇ h ( γ ) = {( \frac{{\partial }^{2} L}{\partial {A}^{2}} )}^{- 1} \frac{{\partial }^{2} L}{\partial \partial A} \qquad Equation 11.$$

Equations 10 and 11 provide the implicit function perspective of how the iterative MLE model 604 builds the functional mapping between measurements and decomposition results. It also sets the reference for explaining why conventional empirical methods suffer from noise amplification. In conventional MD techniques, by carrying out grid calibration, the functional mapping defined by Equation 10 is with paired inputs and outputs, i.e., (γᵢ, Aᵢ), and an empirical model (e.g., a polynomial, a rational polynomial, a neural network, etc.) is fitted to approximate the mapping. That means only the accuracy term (the function value of h(γ̅)) is enforced, while the noise term (∇h(γ̅)) is not included in the fitting. This interpretation explains why conventional empirical models struggle to capture the asymptotically optimal noise performance of iterative MLE MD.

The ideal empirical MD model should “copy” the behavior of the implicit function defined by iterative MLE MD in Equation 10. That means, the objective now becomes finding a function φ(·; θ_{φ}): [symbol] that is an explicit proxy that represents the implicit function φ: [symbol] in both accuracy and noise as shown in Equation 12 below. The desired function φ(·; θ_{φ}), which is that represented by the proxy MD model 606, is thus distinguished with conventional empirical MD methods, with parameters θ_{φ} in the model of Equation 12.

$$φ ( γ ; {θ}_{φ} ) = h ( γ ) , and \frac{\partial φ ( γ ; {θ}_{φ} )}{{\partial }_{γ}} = \frac{\partial h ( γ )}{\partial γ} \qquad Equation 12.$$

In various embodiments, the accuracy and noise requirements can be enforced via Sobolev Learning, which incorporates both target values and target derivatives in the loss as shown in Equation 13, where J and Jₚ are the loss terms for the function value and the p-th order derivatives,

$${D}_{γ}^{p}$$

are the p-th order derivatives with respect to the input γ, and wₚ is the weighting factor for the p-th order loss term, P=1, corresponding to the Jacobian loss.

$${θ}_{φ}^{*} = \begin{matrix}arg min \\ θ φ\end{matrix} {\sum }_{k = 1}^{K} [ J ( φ ( {γ}_{k} ; {θ}_{φ} ) , h ( {γ}_{k} ) ) + {\sum }_{p = 1}^{P} {w}_{p} {J}_{p} ( {D}_{γ}^{p} φ ( {γ}_{k} ; {θ}_{φ} ) , {D}_{γ}^{p} h ( {γ}_{k} ) ) ] \qquad Equation 13.$$

In accordance with some embodiments of training process 600, the universal approximation capability of feedforward neural networks is leveraged to find the neural proxy of Equation 10. With these embodiments, the proxy MD model 606 is a neural network model. In some implementation of these embodiments, the proxy MD model 606 employs a multilayer perceptron (MLP) architecture as the backbone. However, other types of neural network architectures are also applicable. As described above, the training process 600 includes both the value term and first-order derivatives (Jacobians) in the training loss at 620. When only value loss is applied, the fitting is identical to conventional empirical methods. Proxy MD also differs from conventional empirical MD methods by including the forward detector model 408 as input, as shown in FIG. 6 and also mathematically shown by Equation 13.

Both numerical/simulated and experimental studies were carried out to validate the proposed proxy MD as implemented as an MLP. In the numerical validation, two tests of two-basis material decomposition were conducted: a projection-domain test with different combinations of material thickness, and a phantom test in image domain. Water and calcium were selected as the basis materials. In the projection-domain test, sampling points covering water (0:4:36 cm) and calcium (0:0.5:4.5 cm) were selected for proxy MD training and used different thickness combinations for the test. In the phantom test, a 512×512 image was selected from the KITS21 dataset (the 2021 Kidney and Kidney Tumor Segmentation challenge) and water and calcium were mapped based on the Hounsfield Unit (HU) value. The proxy MD model was trained with the same sampling points in accordance with training process 600. A photon-counting detector forward model of a Si-60 mm detector from Monte Carlo simulation was used to generate spectral measurements in 4 energy bins, and a 120 kV spectrum from CatSim was used. The CPU execution time was measured on a workstation (Intel Core i9-9960X CPU @3.1 GHZ). A simple MLP with 2 hidden layers, each with 32 neurons and Tanh activation function, was used as the proxy MD model 606.

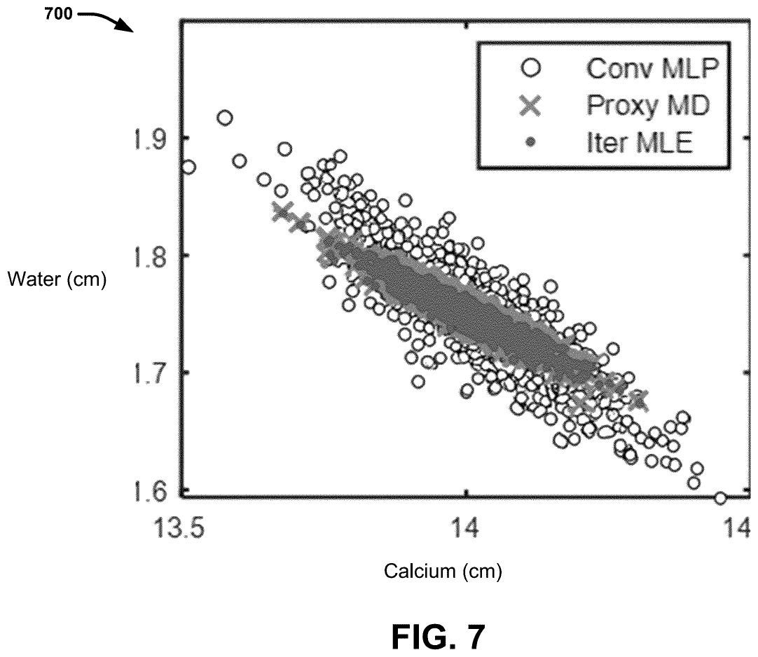

In the physical experiment, the spectral count data was obtained by scanning the Multi-Energy CT head phantom (specifically Gammex) on a whole-body edge-on-irradiated silicon PCCT system. Axial scans were acquired with a tube voltage of 120 kV and a total exposure of 395 mAs. Pixel-level proxy MD models were trained to capture pixel-specific responses from a polyethylene (PE)/polyvinyl chloride (PVC) basis material calibration. There were also two hidden layers in the MLP, each with 64 neurons and Tanh activation function. The results of the simulated/numerical and physical experimental studies are shown in FIGS. 7-11.

FIG. 7 presents a graph 700 comparing the MD results of the proxy MD neural network model with other MD processes (e.g., conventional MLP and iterative MLE) as applied to the physical experiment. In particular, graph 700 shows the material decomposition from noisy measurements at one test point (water=14.5 cm, calcium=1.75 cm), and each point represents the decomposition from one noisy measurement. As shown in FIG. 7, the proxy MD method described herein preserves the desired anti-correlation of iterative MLE MD through Sobolev Learning, thus yielding better noise performance than conventional MLP, which shares the same backbone but is trained with accuracy loss only.

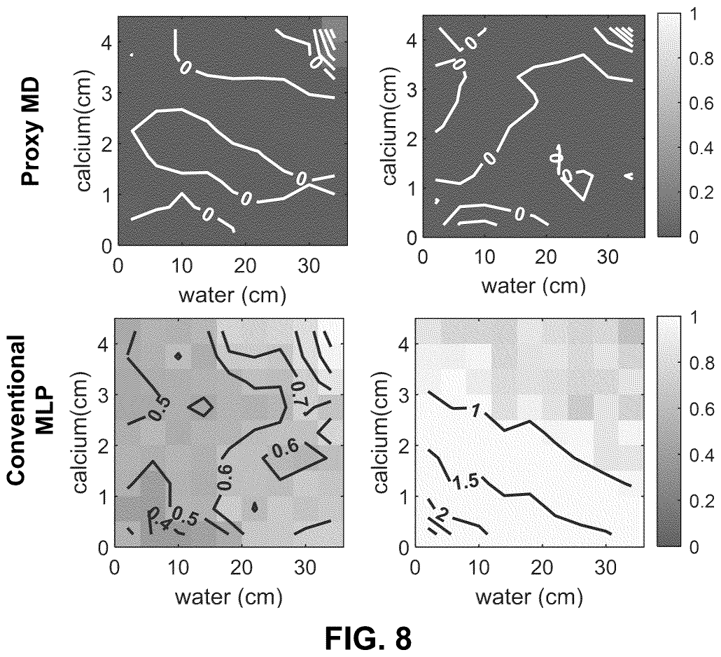

FIG. 8 presents comparative noise amplification results for the proxy MD and the conventional MLP model (trained with accuracy loss only) for the physical experiment. In FIG. 8, the relative noise increase is visualized at all combinations of test points. As shown in FIG. 8, the noise increase is generally zero for proxy MD, meaning there is nearly no noise amplification, while the noise amplification from the conventionally trained MLP is generally more than 50%.

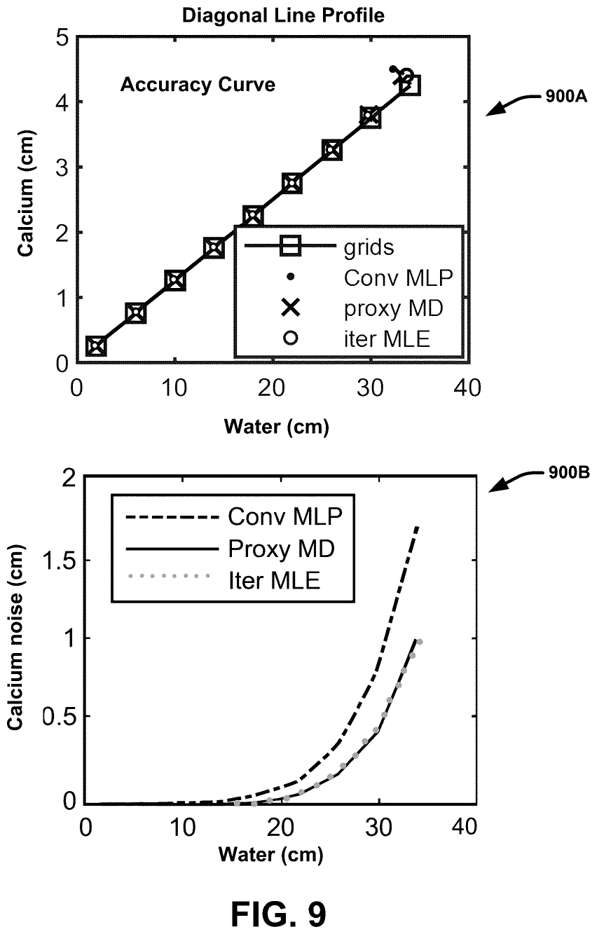

FIG. 9 shows the accuracy curve 900A and noise curve 900B along the diagonal of the test grid for the comparative MD processes evaluated in the physical experiment. FIG. 9 demonstrates that the accuracy performances are good from either proxy MD or conventional MLP, but significant noise amplification occurs in conventional MLP.

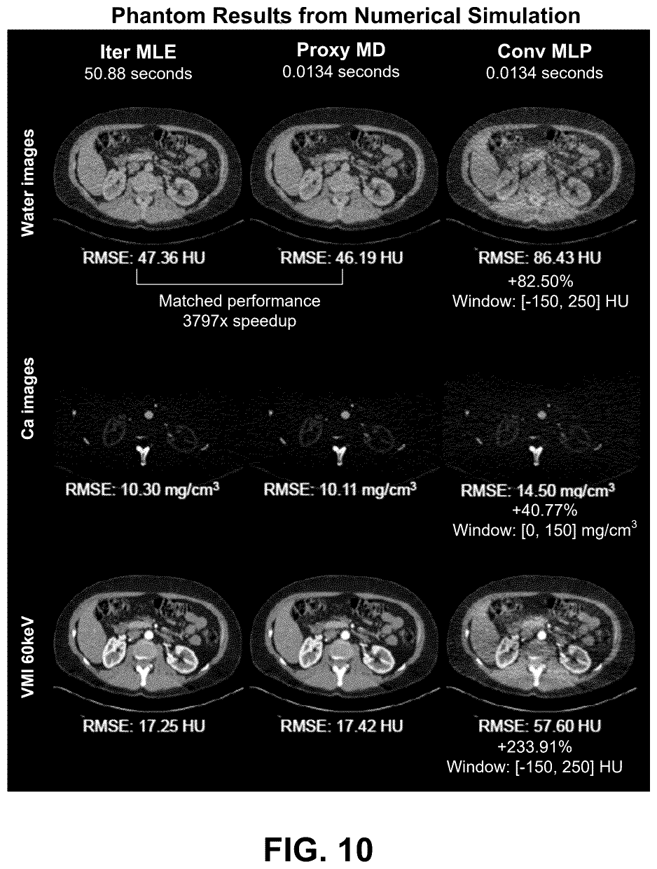

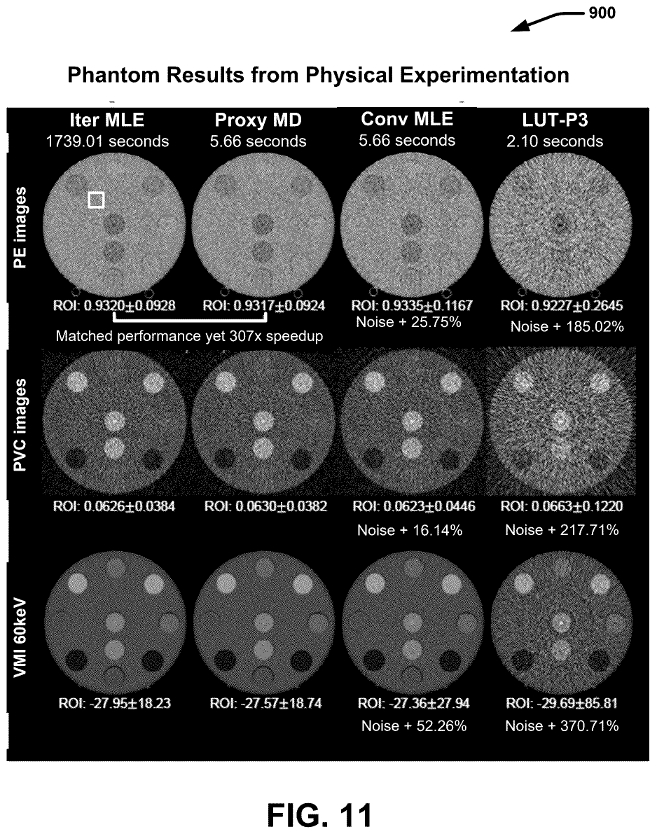

FIG. 10 illustrates the image-domain results of the different MD processes in accordance with the numerical simulation experiment, and FIG. 11 illustrates the image-domain results of the different MD processes in accordance with the physical experiment. The image-domain results of both the numerical simulation align well with the projection-domain findings in terms of accuracy and noise. The root mean square errors (RMSEs) are reported in numerical simulation since access to the clean ground-truth was available. For the physical phantom scan, a region of interest (ROI) was selected and used to evaluate the mean and standard deviation of each method. The corresponding CPU execution time is reported in FIGS. 10 and 11 as well.

In FIG. 10 we see matched RMSE between proxy MD and iterative MLE MD in numerical simulation, showing that the proposed proxy MD method effectively learns the implicit function defined by the iterative solver, while providing >3000 times the speed-up compared to the Nelder-Mead solver (i.e., an iterative MLE MD approach). Conversely, conventional MLP is equivalently fast since it shares the same backbone but suffers from significant noise amplification. This observation is confirmed in the physical experiments and reported in FIG. 11, where the noise level follows the trend. As shown in FIG. 11, the results of proxy MD are substantially equivalent to those of iterative MLE. In addition, iterative MLE comparatively outperforms conventional MLE and LUT-P3 for basis images and VMIs (60 kvV). LUT-P3 is a look-up table based on third-order polynomials, which is another conventional empirical MD method that is fast, but results in even higher noise levels than conventional MLP.

With reference again to FIG. 4, in summary, the differentiable MD operator 414 corresponds to a differentiable version of an iterative MLE solver that performs a differentiable MD process at the material decomposition phase of the pipeline 304. The disclosed subject matter provides three methods for making the material decomposition process of the iterative solver of MLE MD differentiable. In the first method, the MD process is made differentiable by unrolling the iterative MLE solver into a differentiable computational graph. In the second method, the MD process is made differentiable by determining analytical derivatives of the iterative MLE solver using implicit differentiation. In the third method, the MD process is made differentiable by using the proxy MD model 606 as the differentiable MD operator 414, that receives spectral data (or more particularly, PP spectral count data 410) and the forward detector model 408 as input, and generates the material decomposition values (MD data 416) as output, as a result of a training process (e.g., training process 600) used to train the proxy MD model 606. As described above, this training process comprises training the proxy MD model 606 (a differentiable empirical model) to minimize a composite loss function comprising: an accuracy loss term penalizing (first) differences between training material decomposition values output by the proxy MD model 606 based on training spectral count data and reference material decomposition values generated by the iterative MLE solver based on the training input spectral count data; and a derivative loss term penalizing second differences between derivatives of the proxy MD model 606 and reference derivatives of the MLE solver.

Each of the foregoing methods offers distinct advantages in terms of computational performance, numerical stability, and fidelity of the resulting material decomposition. In particular, the unrolled MLE approach advantageously preserves the interpretability and convergence behaviour of the original iterative MLE solver while enabling control over the number of optimization steps to achieve a balance between reconstruction accuracy and computation time. The implicit differentiation method provides a compact and mathematically rigorous mechanism for determining derivatives of the iterative solver without requiring unrolling, thereby reducing memory consumption and avoiding accumulation of truncation errors that can arise from partial unrolling. The proxy MD approach, in turn, provides the benefit of substantially accelerating the MD phase by learning an efficient surrogate that approximates the mapping of the iterative solver with high accuracy, while also enabling improved noise robustness through data-driven regularization learned during training. Collectively, these methods offer complementary strategies that improve the efficiency, stability, and precision of material decomposition within spectral CT imaging workflows.

Furthermore, by replacing the iterative MLE MD solver with a corresponding differentiable MD operator 414 and using differentiable operators in the preprocessing module 404 and the image reconstruction module 418, the whole pipeline 304 becomes differentiable. This configuration allows quantitative information from multiple domains, including the projection, material decomposition, and image domains, to be distilled jointly for training, calibration, and adaptive parameter updates in a data-driven manner. In this framework, gradients of quantitative loss functions computed in the image or projection domain can be propagated upstream through the differentiable reconstruction operator 420, the differentiable MD operator 414, the forward detector mode 408 and the one or more differentiable preprocessing operators 406_{1-N}. As a result, parameters associated with the forward detector model 408 (e.g., energy bin thresholds or gain coefficients), preprocessing transforms performed by the one or more differentiable preprocessing operators 406_{1-N} (e.g., spectral denoising weights, normalization constants, scatter-correction kernels, etc.), and other calibration factors can be automatically adjusted in a data-driven manner. This process results in a calibrated version of the image processing pipeline, thereby improving quantitative image fidelity and system stability without the need for manual recalibration.

In this regard, in conventional spectral CT systems, calibration of the image processing chain is typically performed through a series of manual or semi-automated procedures that rely on reference scans, lookup tables, and parameter sweeps to adjust detector and system characteristics. For example, calibration of the forward detector model may involve repeatedly scanning uniform phantoms to estimate energy bin thresholds, gain factors, or charge-sharing corrections, while preprocessing calibration may require tuning normalization coefficients or scatter-correction parameters based on empirical fits. These procedures are time-consuming, require operator intervention, and must be repeated periodically to compensate for detector aging, temperature drift, or changes in acquisition settings. Moreover, such manual calibrations are generally performed in isolation for each subsystem, without considering cross-domain dependencies between detector response, material decomposition, and reconstruction accuracy. The differentiable, end-to-end calibration framework described herein overcomes these limitations by jointly optimizing all components of the imaging chain using quantitative image-domain feedback, thereby providing a unified and adaptive approach to maintaining calibration accuracy over time.

FIG. 12 illustrates an example method 1200 for calibrating parameters of the differentiable image processing pipeline 304 based on image-domain and/or projection domain results, in accordance with one more embodiments. The solid-line arrows in FIG. 12 represent the forward process or execution of the pipeline 304 by the execution component 306. The dashed-line arrows represent the backpropagation process performed by the calibration/training component 308.

In accordance with the forward process, the raw spectral data 402 acquired by the is sequentially processed through each stage of the differentiable image processing pipeline 304. In association with performing calibration of the pipeline, the raw spectral data 402 may correspond to spectral measurements acquired from one or more calibration phantoms (e.g., water, calcium, or iodine phantoms), uniform flood-field scans, or other reference acquisitions having known material composition and geometry. Such calibration datasets provide a controlled basis for computing quantitative losses in the image or projection domain and for propagating corresponding gradients through the differentiable operators to update detector and system parameters. In some embodiments, once an initial calibration has been performed using phantom or reference data, the differentiable pipeline may be further fine-tuned adaptively using clinical or patient imaging data, thereby continuously maintaining calibration accuracy under actual imaging conditions.

In accordance with method 1200 (and as described with reference to FIG. 4), the preprocessing module 404 includes one or more differentiable preprocessing operators 406_{1-N}, each configured to perform a differentiable transformation on the raw spectral data 402. Example differentiable preprocessing operations include logarithmic transformation, spectral denoising, energy-dependent normalization, and scatter- or beam-hardening correction, each parameterized by learnable weights, scaling factors, or filter coefficients. The preprocessing module generates preprocessed (PP) spectral count data 410, which are provided to the material-decomposition module 412 along with the forward detector model 408 that represents the spectral response characteristics of the Photon Counting detector (e.g., threshold settings, detector gain, and bin-to-energy mapping). Within the material decomposition module 412, the differentiable MD operator 414 estimates basis-material coefficients to produce MD data 416, which are subsequently processed by the image reconstruction module 418 that includes a differentiable reconstruction (Diff. Recon.) operator 420 to generate one or more CT images 422 (e.g., MD images and/or VMIs) and/or corresponding projection images 424. The differentiable MD operator 414 may be implemented in accordance with any of the three methods described herein (e.g., unrolling the iterative MLE solver, implicit differentiation, or as the proxy MD model 606).

In accordance with the backpropagation process, in some embodiments, at 1206 the CT images 422 are compared to reference CT images 1202 and/or the projection images 422 are compared to reference projection images 1204, and the calibration/training component 308 determines a corresponding image-domain loss and/or projection-domain loss using a suitable loss function (e.g., mean square error (MSE), structural similarity index (SSIM), or a composite quantitative loss combining intensity and gradient terms). In various embodiments, the reference data (e.g., the reference CT images 1202 and the reference projection images 1204) may correspond to ground-truth or otherwise known target representations of the imaged object or calibration phantom. For example, in a phantom-based calibration mode, the reference CT images 1202 can include material decomposition maps or attenuation images generated from analytical models, prior calibrations, or high-fidelity reference scans (e.g., dual-energy CT or extended dynamic range PCCT acquisitions) of the same phantom under known conditions. Similarly, the reference projection images 1204 can include simulated or experimentally measured spectral projection data generated based on a known forward model or on previously validated system calibration parameters. In a clinical or adaptive fine-tuning mode, the reference data may alternatively correspond to baseline images from a prior scan, multi-energy reconstruction, or model-based estimation of material composition derived from prior patient-specific imaging records. In each case, the reference data serves as quantitative supervision targets for computing the loss function, such that minimizing the loss aligns the reconstructed and reference representations in intensity, material composition, or other quantitative metrics of interest.

In other embodiments, at 1206, the image-domain loss used for calibration or training may be defined without explicit reference images, relying instead on regularization or prior-based constraints applied directly to the reconstructed images or intermediate material maps. Such unsupervised or self-supervised loss functions may include, for example, total-variation (TV) regularization, spatial-smoothness penalties, sparsity constraints, or edge-preserving priors that encourage physical plausibility and noise suppression in the reconstructed images. These formulations allow the differentiable image processing pipeline 304 to perform calibration or adaptive fine-tuning in scenarios where reference ground-truth data are unavailable, while still providing meaningful gradient signals for updating parameters of the preprocessing operators, the forward detector model 408, or other differentiable components.

At 1208, the computed loss is then used to evaluate gradients of the loss function with respect to the parameters of the differentiable reconstruction operator 420, the differentiable MD operator 414, the differentiable preprocessing operators 406_{1-N}, and/or the forward detector model 408, thereby quantifying the sensitivity of the image-domain error to upstream components of the imaging chain. These gradients are subsequently propagated backward through the differentiable pipeline to update the corresponding parameters, resulting in an adaptively calibrated version of the pipeline 304.

More particularly, at 1208 gradients of the loss determined at 1208 are propagated backward through the differentiable reconstruction operator 420, followed by the differentiable MD operator 414, and then followed by the differentiable preprocessing operators 406 and/or the forward detector model 408 to update one or more parameters of the corresponding differentiable operators. This gradient backpropagation corresponds to computing partial derivatives of the loss with respect to parameters of upstream operators, thereby quantifying how changes in those parameters affect the final image output.

The numbered circles along the dashed-line paths in FIG. 12 denote examples of upstream parameter-update stages. Circle \textcircled{1} corresponds to gradient backpropagation through the differentiable reconstruction operator 420. For example, based on the gradients of an image-domain loss propagated backward through the differentiable reconstruction operator 420, the calibration/training component 308 may update one or more parameters of the reconstruction model, such as, but not limited to, regularization weights, kernel coefficients, or iterative-solver step sizes.

For instance, in one embodiment, the differentiable reconstruction operator 420 may be implemented as a neural network based reconstruction model, such as a convolutional neural network (CNN), a U-Net, or a learned iterative reconstruction network. In this configuration, the differentiable reconstruction operator 420 learns a mapping between material-decomposition data and quantitative image space. During calibration, gradients of the image-domain loss (e.g., mean-square error, structural similarity, or perceptual loss) are automatically back-propagated through the network layers to update trainable parameters such as convolutional-kernel weights, bias terms, or feature-normalization coefficients using a gradient-descent optimizer. The resulting updates allow the differentiable reconstruction operator 420 to adaptively learn noise-suppression and artifact-correction behaviors that improve quantitative image fidelity.

In another embodiment, the differentiable reconstruction operator 420 may correspond to a differentiable analytical or iterative reconstruction algorithm, such as a filtered back-projection (FBP) function, a differentiable proximal-gradient method, or a differentiable model-based iterative reconstruction (MBIR) solver. In such embodiments, backpropagation involves computing derivatives of the reconstruction output with respect to algorithmic parameters, such as derivatives of the FBP filter response with respect to cutoff frequency, derivatives of iterative-solver updates with respect to step-size or convergence weights, or derivatives of the regularization term with respect to spatial or spectral weighting coefficients. These gradients are then used by the calibration/training component 308 to refine the reconstruction parameters so that reconstructed images minimize the quantitative loss when compared with reference images or known physical priors.

Accordingly, gradient backpropagation through the differentiable reconstruction operator 420 enables automated calibration of both learned and analytical reconstruction functions, improving quantitative accuracy and ensuring that the reconstruction stage remains optimally tuned to upstream detector and material-decomposition characteristics.

Circle \textcircled{2} represents propagation through the differentiable MD operator 414, which facilitates parameter updates within the MD operator itself, such as for example, material-basis coefficients, spectral mixing matrices, or regularization hyperparameters. As illustrated by the dashed-line paths in FIG. 12, the gradients propagated from the differentiable reconstruction operator 420 (Circle \textcircled{1}) are transmitted backward into the MD stage, linking reconstruction-domain sensitivity to the upstream spectral decomposition process. In this manner, changes in reconstructed-image fidelity directly inform how the differentiable MD operator 414 adjusts its internal parameters so that material decomposition outputs better support quantitative image accuracy in subsequent reconstruction.

In particular, when the differentiable reconstruction operator 420 outputs image-domain gradients ∂L_{img}/∂I_{A}, those gradients are back-propagated through the differentiable reconstruction operator's Jacobian with respect to its inputs, yielding gradients ∂L_{img}/∂A in the material decomposition domain. These MD-domain gradients serve as the input signals for Circle \textcircled{2} and are applied to update parameters of the MD operator 414 according to its differentiable formulation. Consequently, the calibration/training component 308 uses the reconstruction-level feedback to refine the differentiable MD operator 414 so that the decomposition outputs A′yield reconstructed images that minimize the image-domain loss.

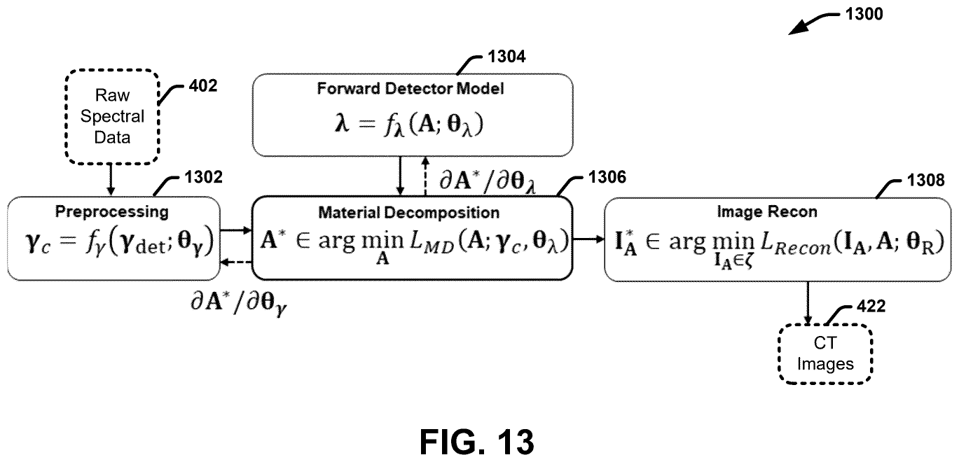

In some embodiments, the differentiable MD operator 414 is implemented as an unrolled iterative solver, in which each iteration of an MLE-based decomposition process is represented as a differentiable computational step. In this configuration, the gradients ∂L_{img}/∂A are recursively propagated through the sequence of solver iterations in accordance with Equation 18 (see FIG. 13). The calibration/training component 308 applies these propagated gradients to update model parameters such as material-basis scaling factors, spectral-mixing coefficients, or iteration-step weights, thereby learning an optimal decomposition process that minimizes the propagated reconstruction loss.

In another embodiment, the differentiable MD operator 414 is implemented using implicit differentiation of the MLE solver. In this embodiment, the converged decomposition solution A*is treated as an implicit function of model parameters, and the total derivatives of A* with respect to detector-model and preprocessing parameters are computed analytically in accordance with Equation 19 (see FIG. 13). The image-domain gradients ∂L_{img}/∂A are combined with these analytical derivatives to yield parameter-specific gradients ∂L_{img}/∂θ_{λ} and ∂L_{img}/∂θ_{γ}, which are then used to update one or more parameters of the forward detector model 408 and/or the one or more differentiable preprocessing operators 406_{1-N}. This approach enables efficient and stable backpropagation through the MD operator without explicitly unrolling the iterative solver.

In still another embodiment, the differentiable MD operator 414 corresponds to a trained version of the proxy MD model 606 that has been trained to approximate the mapping of the iterative MLE solver. In this configuration, the gradients ∂L_{img}/∂A propagated from the reconstruction stage are back-propagated through the network layers using conventional neural network differentiation. During calibration, the network weights and bias terms may be updated together with upstream detector model and preprocessing parameters so that the learned proxy MD model emulates both the forward mapping and the sensitivity characteristics of the iterative solver. This data-driven approach enables end-to-end training of the entire imaging chain with substantially reduced computational overhead compared with explicit iterative optimization.

Accordingly, circle \textcircled{2} represents the gradient backpropagation and parameter-update phase of the differentiable MD operator 414, through which reconstruction-domain loss information is translated into adjustments of spectral decomposition parameters. This ensures that the material-decomposition stage remains quantitatively consistent with downstream reconstruction objectives and cooperatively optimized with other differentiable components of pipeline 304.

Circle \textcircled{3} corresponds to gradient propagation through the forward detector model 408, allowing correction of detector-specific parameters such as energy bin thresholds, per-bin gain factors, charge-sharing coefficients, or pulse pile-up compensation parameters. In the differentiable pipeline, gradients reaching the forward detector model 408 originate from the differentiable MD operator 414 (Circle \textcircled{2} and represent the sensitivity of the image-domain loss with respect to the detector-model outputs and their underlying parameters. The calibration/training component 308 applies these gradients to update detector-model parameters θ_{λ} so that the predicted spectral responses f_{λ}(A; θ_{λ}) more accurately match the statistical and spectral characteristics of the measured photon counts.

For example, when the forward detector model 408 is implemented as an analytical or physics-based differentiable model, backpropagation involves computing partial derivatives of the expected spectral counts with respect to the detector-model parameters for each energy bin. These derivatives quantify how small adjustments in the detector thresholds or gains affect the expected counts. The calibration/training component 308 then updates the corresponding parameters using gradient-based optimization to correct spectral shifts, bin-drift errors, or other detector non-idealities.

In another embodiment, the forward detector model 408 may be implemented as a data-driven differentiable model, such as a neural network-based detector-response model trained to approximate the physical forward mapping between material projections and measured photon counts. In this case, gradients of the image-domain loss are propagated through the network by standard backpropagation to adjust its learnable parameters (e.g., network weights, bias terms, or learned threshold representations). This embodiment allows the forward detector model 408 to adaptively learn complex or non-linear detector behaviors that are difficult to capture analytically, while remaining coupled to the overall quantitative imaging objective.

Circles \textcircled{4}, \textcircled{5}, and N correspond to propagation through the differentiable preprocessing operators 406_{1-N}, allowing updates to denoising filter weights, normalization constants, bias-field-correction parameters, scatter correction parameters, and other preprocessing parameters that influence the statistical and spectral quality of the spectral count data. During calibration, the gradients propagated from the detector-model stage are applied to the preprocessing operators 406_{1-N} via the calibration/training component 308, which adjusts the associated parameters so that the preprocessed spectral count data 410 (e.g., γ_{c}=f_{γ}(Y_{det}; θ_{γ}) remains statistically consistent with the calibrated detector responses and the reconstructed quantitative images.

In one embodiment, a preprocessing operator 406₁ for example, may be implemented as a neural network-based differentiable function, such as a convolutional denoising autoencoder or a residual CNN trained to perform a preprocessing task to transform the raw spectral data 402 into the PP spectral count data. For example, the preprocessing task may correspond to suppressing Poisson noise and spectral cross-talk, reducing/correcting scatter, or another preprocessing task. In this configuration, the network receives the detector-domain spectral counts Y_{det} as input and outputs improved or corrected spectral data γ_{c}. Gradients of the image-domain loss function are automatically propagated backward through the network to update learnable parameters such as convolutional-kernel weights, bias terms, or normalization coefficients. As a result, the neural preprocessing operator learns data-driven corrections for non-linear detector artifacts and statistical noise characteristics in a manner directly tied to the quantitative imaging objective. With these embodiments, method 1200 may be used to train the neural network-based preprocessing operator based on backpropagation of gradients of the image-domain loss through the upstream differentiable operators and to the preprocessing model, thereby eliminating the need for ground truth corrected spectral data γ_{c(ref)} while further tailoring the preprocessing model optimization based on ideal representations of the final target, that is the CT images themselves.

In another embodiment, a preprocessing operator 406₁ for example, may be implemented as a differentiable analytical transformation, such as a spectral-response normalization function, a polynomial bias-field correction, or a differentiable scatter-correction model. For example, the operator may apply a pixel-wise normalization in accordance with Equation 14.

$${γ}_{C} = α ( {Y}_{det} ) \cdot {Y}_{det} + β , \qquad Equation 14.$$

In Equation 14, α and β are differentiable functions parameterized by θ_{γ} (e.g., smooth weighting functions or learnable scaling parameters). During calibration, gradients of the quantitative loss are back-propagated through this function to refine the normalization or scatter-correction parameters, ensuring that the corrected counts maintain spectral fidelity and consistency with the forward detector model.

Accordingly, gradient propagation through the differentiable preprocessing operators 406_{1-N} enables both data-driven and analytical preprocessing operators to be adaptively tuned in concert with the forward detector model 408, the MD differentiable operator 414, and differentiable reconstruction operator 420, thereby improving the statistical accuracy and spectral integrity of the Photon Counting CT imaging chain.

Through this coordinated process, the calibration/training component 308 performs end-to-end optimization of the differentiable image processing pipeline 304, applying gradients of the quantitative loss function determined at 1206 to each differentiable stage of the imaging chain. The result is an adaptively calibrated spectral CT system in which detector, preprocessing, material decomposition, and reconstruction parameters are jointly optimized under supervision of quantitative image-domain or projection-domain targets. This enables automatic correction of detector bin-drift and gain errors, compensation for spectral distortions, and improved consistency between measured spectral counts and reconstructed quantitative images, thereby enhancing both image fidelity and system stability.

FIG. 13 illustrates an example mathematical framework 1300 for gradient backpropagation in a differentiable material decomposition (MD) operator when implemented using either an unrolled iterative solver or an implicit-differentiation method. Framework 1300 depicts the data and gradient dependencies among the differentiable preprocessing operators, forward detector model, differentiable material decomposition operator and differentiable image-reconstruction components of a pipeline 304 in embodiments in which the differentiable material MD operator 414 is implemented using either an unrolled iterative solver or an implicit-differentiation method.

Framework 1300 includes a preprocessing operator 1302 (which corresponds to an example preprocessing operator of amongst preprocessing operators 406_{1-N}), a forward detector model 1304 (which corresponds to an example of the forward detector model 408), a material decomposition operator 1306 (which corresponds to an example of the differentiable MD operator as implemented using either an unrolled iterative solver or an implicit-differentiation method), and a differentiable image reconstruction operator 1308 (which corresponds to an example of the differentiable image reconstruction operator 420).

The preprocessing operator 1302 is mathematically represented by example Equation 15.

$${γ}_{C} = {f}_{γ} ( {Y}_{det} ; {θ}_{γ} ) \qquad Equation 15.$$

In Equation 15, Y_{det} denotes the raw spectral data 402 acquired from the detector elements; f_{γ}(·) denotes a differentiable preprocessing function or set of operations (e.g., normalization, gain correction, denoising, scatter correction, or bias-field correction); and θ_{γ} represents a vector of preprocessing parameters such as normalization constants, denoising-filter weights, or bias-field coefficients. The output γ_{c} represents the preprocessed spectral count data provided to the subsequent material decomposition operator 1306.

The forward detector model 1304 is mathematically represented by example Equation 16.

$$λ = {f}_{λ} ( A ; {θ}_{λ} ) \qquad Equation 16.$$

In Equation 16, λ represents the expected or “clean” spectral data predicted for each energy bin; A represents the vector of material-decomposition coefficients (e.g., line integrals or areal densities of basis materials such as water, iodine, or calcium); f_{λ}(·) denotes a differentiable detector-response model; and θ_{λ} represents detector-model parameters such as energy-threshold settings, gain factors, bin-drift offsets, and charge-sharing or cross-talk coefficients.

The material decomposition operator 1306 is mathematically represented by Equation 3 (previously introduced with reference to FIG. 4).

$${A}^{*} = arg {min}_{A} {L}_{MD} ( A ; {γ}_{C} , {θ}_{λ} ) \qquad Equation 3.$$

In Equation 3, L_{MD} denotes the material decomposition loss function (for example, a maximum-likelihood-estimation (MLE) objective that models Poisson statistics of photon counts), A*denotes the estimated material decomposition coefficients that minimize this loss, γ_{C} denotes the preprocessed spectral counts, and θ_{λ} denotes the detector-model parameters. Equation 3 therefore represents an iterative MLE process that is solved numerically to obtain A*.

The differentiable image reconstruction operator 1308 is mathematically represented by Equation 17.

$${I}_{A}^{*} = arg {min}_{{I}_{A} ∈ ζ} {L}_{Recon} ( {I}_{A} , A ; {θ}_{R} ) \qquad Equation 17.$$

In Equation 17,

$${I}_{A}^{*}$$

denotes the reconstructed quantitative or CT images corresponding to the basis-material maps; L_{Recon} represents a differentiable reconstruction loss (for example, a regularized least-squares or data-fidelity objective); θ_{R} represents reconstruction parameters such as regularization weights or convolution-kernel coefficients; and ζ represents the domain of permissible reconstructed images.

The solid arrows in FIG. 13 represent the forward flow of data from detector measurements to reconstructed images. The dashed arrows ∂A*/∂θ_{λ} and ∂A*/∂θ_{γ} represent gradient dependencies of the MD solution A*on the detector-model and preprocessing parameters, respectively. These gradients are used during backpropagation to propagate image-domain loss upstream through the differentiable MD operator and to update system parameters.

In the unrolled-solver embodiment, the iterative updates of the MLE solver are explicitly represented as differentiable computational steps. Gradients with respect to the model parameters θ can be computed by differentiating through each iteration according to the recursive relation represented in Equation 18.

$$\frac{\partial {A}_{n}}{\partial θ} = \frac{\partial ϕ ( {A}_{n - 1} ; θ )}{\partial {A}_{n - 1}} \frac{\partial {A}_{n - 1}}{\partial θ} + \frac{\partial ϕ ( {A}_{n - 1} ; θ )}{\partial θ} \qquad Equation 18.$$

In Equation 18, φ(Aₙ₋₁; θ) denotes the iterative update rule for the MLE solver at iteration n; An represents the material-coefficient estimate after the n^{th} update; and the two terms represent, respectively, (1) the propagation of sensitivities through the previous iteration and (2) the direct dependence of the update rule on the parameters θ. In this regard, Equation 18 corresponds to the unrolled method (also represented via Equation 5) in which the entire optimization sequence is unfolded into a differentiable graph, allowing gradients to be computed by standard automatic-differentiation frameworks. In particular, during unrolled backpropagation, gradients of the material-decomposition solution with respect to the detector-model parameters θ_{λ} and the preprocessing parameters θ_{γ} are obtained as ∂A*/∂θ_{λ} and ∂A*/∂θ_{γ}, respectively, by recursively applying Equation 18 through each iteration of the solver.

In the implicit-differentiation embodiment, the MLE solver is treated as an implicit function that yields the converged solution A* satisfying the stationary-point condition ∂L_{MD}/∂A|_{A*}=0. Using the implicit-function theorem, the total derivative of the solution with respect to any parameter θ can be expressed as Equation 19.

$$\frac{\partial {A}^{*}}{\partial θ} = - {( \frac{{\partial }^{2} {L}_{MD}}{\partial {A}^{2}} )}^{- 1} \frac{{\partial }^{2} {L}_{MD}}{\partial θ \partial A} \qquad Equation 19.$$

In Equation 19, H=∂²L_{MD}/∂A² denotes the Hessian of the material-decomposition loss with respect to the decomposition variables A. The term ∂²L_{M}D/∂θ ∂A represents the cross-partial derivatives of the loss with respect to the model parameters θ and the decomposition variables. In particular, the gradient applied to the Photon Counting detector model corresponds to

$$\frac{\partial {A}^{*}}{\partial {θ}_{λ}} ,$$

and the gradient applied to the preprocessing model corresponds to

$$\frac{\partial {A}^{*}}{\partial {θ}_{λ}} .$$

Each of these gradients may be determined according to Equation 19, wherein θ_{λ} and θ_{γ} represent, respectively, the parameters of the detector model and the preprocessing operator. This analytical formulation computes gradients of the converged MD solution without unrolling the iterative process, providing efficient and numerically stable gradient computation that is well suited for detector-pixel-specific modeling and calibration.

Accordingly, FIG. 13 mathematically illustrates two alternative approaches, unrolled and implicit differentiation, for computing gradients of the MD operator that enable end-to-end differentiability of the spectral CT imaging pipeline.

In other embodiments, the material decomposition operator 1306 may be implemented as a trained version of the proxy MD model 606 that approximates the mapping of the iterative MLE solver. In this case, proxy MD model 606 directly receives preprocessed spectral data γ_{C} and detector-model parameters θ_{λ} as inputs and outputs estimated decomposition coefficients A*. During training or calibration, gradients of an image-domain loss are back-propagated through the proxy-MD network by conventional neural-network differentiation of network weights, rather than by explicit differentiation of the MLE objective. The resulting gradients are further propagated upstream through the preprocessing and detector models using the same dependency pathways illustrated in FIG. 13. Thus, the proxy-MD embodiment maintains the same overall forward and backward information flow as the unrolled and implicit-differentiation embodiments while replacing the explicit analytical gradient computation with a data-driven neural-network approximation learned from representative spectral-count and material decomposition data.

Experimental implementations of process 1200 (and/or framework 1300) were conducted to demonstrate the cross-domain optimization capabilities of the differentiable image processing pipeline 304, including forward detector model calibration and preprocessing operator training/calibration.

In one experimental implementation, process 1200 (and/or framework 1300) was applied to calibrate a simulated version of the forward detector model 408 in association with correcting detector bin drift. In this implementation, a Photon Counting detector model was simulated with 256 detector pixels and four discrete energy bins. To emulate calibration error, three of the pixels were assigned a bin drift of approximately ±1.5 keV in one of their bins, producing characteristic ring artifacts in the reconstructed images. A water-and-calcium elliptical phantom was used to perform calibration, while a patient dataset was used for subsequent testing and validation.

During calibration, the differentiable image processing pipeline 304 was executed end-to-end, including differentiable preprocessing, differentiable material-decomposition (MD), and differentiable image-reconstruction operators. The forward detector model 408 in this experiment represented a parametric model of detector response, characterized by a set of learnable parameters θ_{λ}, including: (1) energy bin threshold parameters defining the boundaries between the detector's discrete energy channels; (2) per-bin gain and offset factors controlling scaling of the detected counts; and (3) optional spectral-response terms accounting for charge-sharing or pulse-pile-up effects.

The loss function was defined in the material-image domain as a quantitative difference between reconstructed water and calcium material-decomposition images and corresponding reference material maps derived from the phantom.

Gradients of this material-domain loss were propagated backward through the differentiable reconstruction operator 420 and the differentiable MD operator 414 to the detector model 408. The calibration/training component 308 applied these gradients to iteratively update the detector parameters θ_{λ}, adjusting the energy bin thresholds and gain coefficients to minimize the reconstruction loss. Each gradient-descent step effectively corrected small deviations in the detector response model such that the predicted spectral counts f_{λ}(A; θ₈₀) became consistent with the actual measured counts after compensation for drift.

After convergence, the calibrated detector model

$${f}_{λ} ( A ; {θ}_{λ}^{( cal )} )$$

represented a physically corrected spectral-response function for each pixel and energy bin. This calibrated model was then re-used in the pipeline 304 (e.g., in the forward projection and material-decomposition stages) to generate corrected material-decomposition and CT images based on new raw spectral count data acquired from a patient scan. For comparison, “uncorrected” images were reconstructed from the same input spectral-count data using the original, uncalibrated detector model that still contained bin-drift offsets. In contrast, the “corrected” images were reconstructed using the calibrated detector model with updated energy bin thresholds and gain parameters. The calibrated model reduced the mean bin-energy error to less than approximately 0.03 keV and effectively eliminated the ring artifacts associated with mis-calibrated detector pixels.

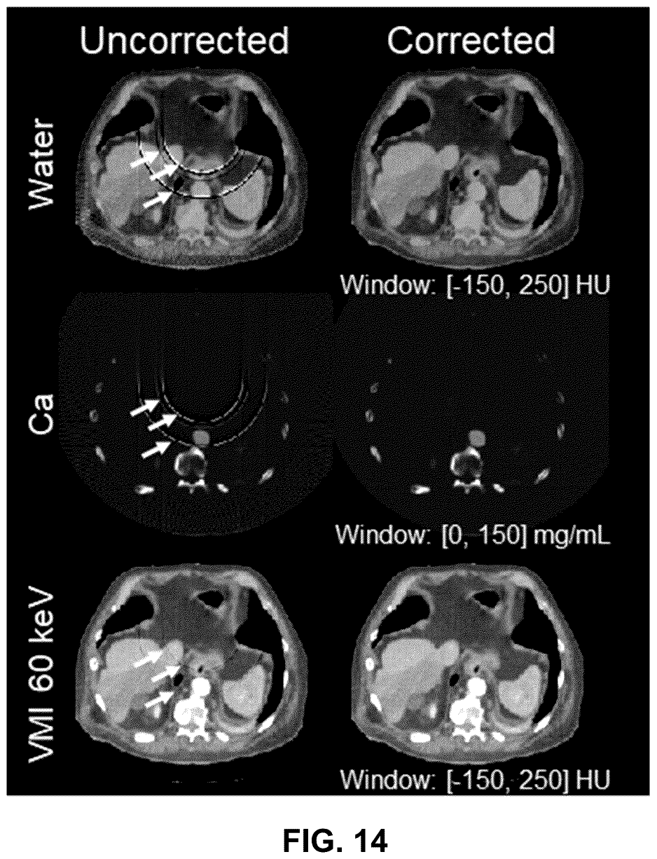

FIG. 14 shows representative results of this experiment, where the uncorrected water, calcium, and virtual monochromatic (VMI) images exhibit circular banding artifacts (indicated by arrows) caused by pixel-specific threshold drift. The corresponding corrected images generated using the calibrated detector model demonstrate restoration of spatial uniformity and accurate quantitative contrast, confirming that the differentiable Photon Counting CT (PCCT) framework successfully identified and compensated for detector-bin drift using only image-domain supervision.

This experiment confirms that the differentiable PCCT framework enables physics-informed calibration of detector-level parameters through gradient backpropagation from the image domain. By optimizing the forward detector model jointly with the MD and reconstruction operators, the system automatically corrected bin-threshold offsets and restored spectral uniformity without explicit detector-level recalibration measurements. The same differentiable approach may be extended to compensate for other detector non-idealities such as gain variation, charge-sharing, or spectral-response distortion, thereby improving system stability and quantitative accuracy in spectral CT imaging.

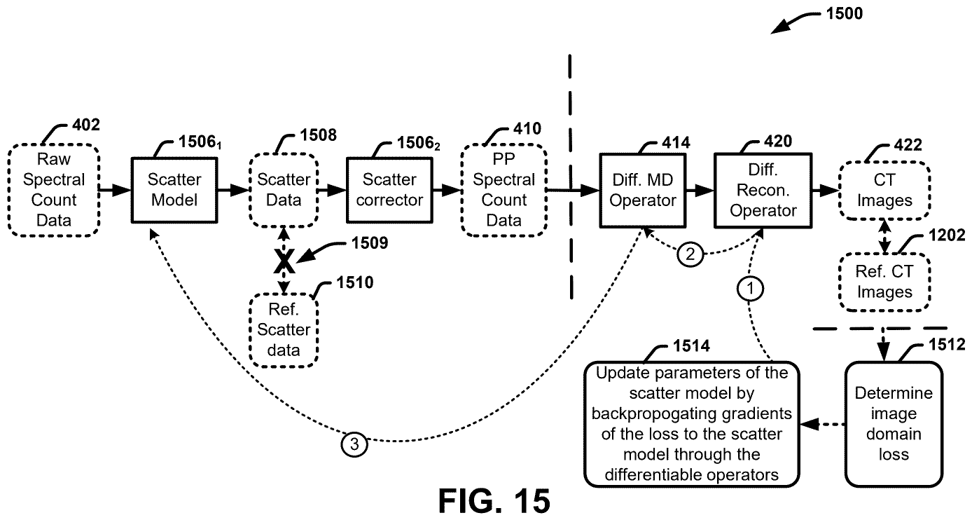

FIG. 15 illustrates an example process 1500 for training a scatter correction model of the disclosed end-to-end differentiable image processing pipeline 304 based on image-domain loss, in accordance with one or more embodiments. FIG. 15 illustrates an abbreviated representation of the differentiable image processing pipeline 304 for ease of illustration. Repetitive description of like elements employed in respective embodiments is omitted for sake of brevity.

In FIG. 15, scatter model 1506₁ and scatter corrector 1506₂ respectively correspond to example differentiable preprocessing operators (e.g., corresponding to the one or more differentiable preprocessing operators 406_{1-N}) that may be included in the preprocessing module 404, in one or more embodiments. In accordance with this embodiment, the scatter model 1506₁ is a neural network model that is being trained to estimate, based on the raw spectral data 402 as input, scatter data 1508 that represents the fraction of total detected counts attributable to scattered photons for each detector element and energy bin. The scatter corrector 1506₂ is a differentiable operator that generates PP spectral count data 410 corresponding to scatter-corrected spectral count data by modifying the raw spectral data 402 in accordance with the estimated scatter data 1508. For instance, the scatter corrector 1506₂ may compute a corrected signal according to Equation 20.

$${Y}_{corr} = {Y}_{det} \times ( 1 - \hat{S} ) , \qquad Equation 20.$$

In Equation 20, where Y_{det} denotes the measured counts and S denotes the predicted scatter-to-total ratio output by the scatter model. The corrected spectral count data (e.g., PP spectral count data 410) is then processed through the differentiable MD operator 414 and the differentiable reconstruction operator 420 to generate quantitative CT images 422, as previously described.

In accordance with process 1500, and as enabled by inclusion of the differentiable MD operator 414 and differentiable reconstruction operator 420 within the pipeline 304, the calibration/training component 308 trains the scatter model 1506₁ based on image-domain loss determined at 1512. At each training iteration, the calibration/training component 308 computes a loss using a suitable loss function (e.g., mean-square error, structural-similarity index, or perceptual loss), based on differences between the CT images 422 and reference CT images 1202, then backpropagates gradients of this loss through the differentiable reconstruction and MD operators, as indicated by dashed arrows and numbered circles \textcircled{1}-\textcircled{3}. In alternative embodiments, the image-domain loss here may be defined without explicit reference images, relying instead on regularization or prior-based constraints applied directly to the reconstructed images or intermediate material maps. Such unsupervised or self-supervised loss functions may include, for example, TV regularization, spatial-smoothness penalties, sparsity constraints, or edge-preserving priors that encourage physical plausibility and noise suppression in the reconstructed images.

At 1514, the calibration/training component 308 updates one or more parameters of the scatter model 1506₁ (for example, neural-network kernel weights, bias terms, or normalization coefficients) using a suitable optimization process, which in some embodiments comprises a gradient-descent-based method and in other embodiments may include second-order or heuristic optimization techniques. The optimization is repeated across multiple training iterations until a convergence criterion is satisfied, after which the calibrated scatter model 1506₁ is deployed within runtime executions of the pipeline 304 to provide real-time scatter correction during imaging.

By contrast, in a conventional PCCT image-processing pipeline that is not end-to-end differentiable, the image-domain loss cannot be propagated upstream to the preprocessing stage. As shown by the crossed-out arrow 1509 in FIG. 15, such non-differentiable pipelines require explicit reference scatter data 1510 (e.g., derived from Monte-Carlo simulation or separate calibration measurements) to train a scatter model, where the loss is computed based on the difference between the predicted scatter 1508 and the reference scatter 1510. However, generating such reference scatter data is computationally expensive and time-consuming, often requiring extensive Monte Carlo transport modeling for each scanner geometry, acquisition protocol, and object composition. Furthermore, these simulation-based scatter references are sensitive to inaccuracies in assumed material properties and typically fail to generalize across different patient sizes or anatomical configurations. In some cases, physical calibration phantoms are used instead, but these methods cannot easily capture dynamic scatter effects that vary across patient poses and X-ray spectra. In process 1500, these limitations are overcome, because no explicit scatter ground truth is needed. Instead, the differentiable pipeline leverages image-domain supervision from reconstructed CT images to automatically refine the scatter model, enabling self-supervised training that is both computationally efficient and physically adaptive to diverse imaging conditions.

Accordingly, process 1500 illustrates an embodiment in which a differentiable preprocessing operator (e.g., a scatter-correction model) is optimized in a self-supervised, physics-consistent manner via end-to-end differentiable propagation of image-domain loss through the full spectral-CT imaging chain. This approach enables training and/or calibration of diverse preprocessing operators tailored to any preprocessing task, not just scatter correction for example, directly from quantitative image targets, improving accuracy and robustness of spectral CT reconstruction without explicit ground-truth calibration data.

In one experimental implementation of process 1500, a differentiable preprocessing model corresponding to the scatter model 1506₁ was trained to estimate and correct object scatter in Photon Counting spectral CT data. A numerical simulation dataset was generated to emulate a cone-beam flat-panel spectral CT configuration using 15 patient cases. For each patient, 48 projections were simulated over a 240-degree rotation with a 5-degree angular step. Object scatter was simulated using a GPU-enabled Monte Carlo photon-transport model, and the resulting measurements were binned into four energy bins with thresholds of 15, 33, 45, and 59 keV corresponding to a 120 kV source spectrum. The simulated scatter-to-primary ratio (SPR) was varied to represent different degrees of scatter contamination in the spectral counts.

The total dataset comprised 528 projections from 11 patients for training, 96 projections from 2 patients for validation, and 96 projections from 2 patients for testing. The differentiable preprocessing model 1506₁ was implemented as a convolutional neural network (CNN) configured to map the measured photon-count data Y_{det} (384×256×4) to a predicted scatter-to-total ratio S_{pred}(128×4) per energy bin. A Sigmoid layer was used in the final network stage to normalize outputs between 0 and 1, ensuring that the predicted scatter ratio remained physically valid. The scatter-corrected counts were computed as Y_{corr}=Y_{det}×(1−S_{pred}), and were then propagated through the differentiable MD operator 414 and the differentiable reconstruction operator 420 to generate material-decomposition images (e.g., water and calcium basis images) and reconstructed CT images 422.

The scatter model was trained using mean-square-error (MSE) loss defined in the quantitative image domain between reconstructed material-decomposition images and reference images reconstructed from scatter-free data. Loss gradients were backpropagated through the differentiable reconstruction and MD operators to update the weights of the scatter model via gradient descent. Training was performed for 200 epochs using an Adam optimizer with an initial learning rate of 1×10⁻⁴ and a decay factor of 0.1 every 100 epochs. A derivative-aware proxy MD model preserving the Jacobian of the iterative MLE material-decomposition process was used as the differentiable MD operator 414. This end-to-end differentiable framework enabled cross-domain learning of the scatter-correction model directly from image-domain supervision without Monte Carlo-derived scatter references.

After training, the calibrated scatter model was applied to unseen spectral-count data from independent phantom and patient cases to produce scatter-corrected measurements. The corrected data were processed through the differentiable MD and reconstruction pipeline to yield quantitative CT images exhibiting improved contrast linearity, reduced shading, and suppression of scatter-induced bias. The performance of the proposed cross-domain training (denoted Cross-NN) was compared to conventional empirical correction and direct-reference neural-network training (Direct-NN). The empirical model relied on polynomial scatter-ratio fitting, whereas the Direct-NN model required explicit scatter references generated from Monte Carlo simulation. The Cross-NN model achieved equivalent quantitative accuracy while eliminating the need for explicit scatter reference data.

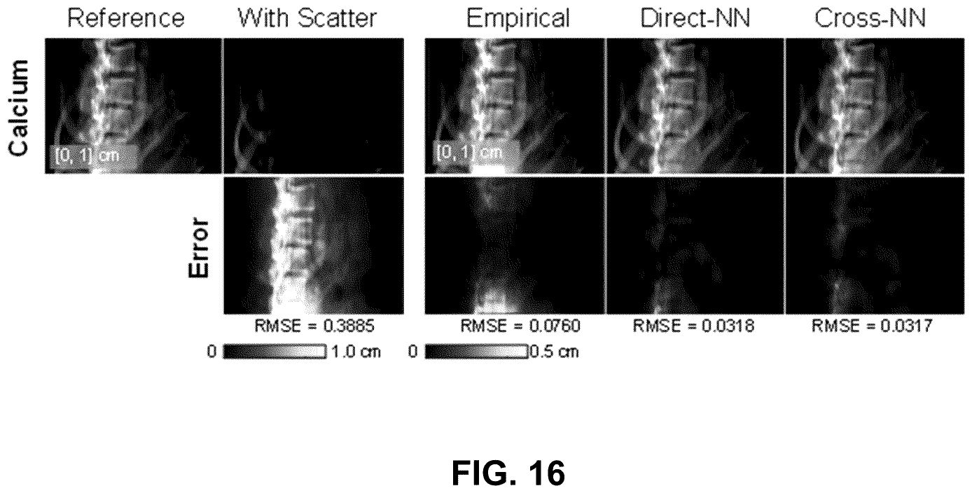

FIG. 16 illustrates representative calcium-basis images and corresponding error maps obtained from the test dataset. The “Reference” image represents the ground-truth decomposition reconstructed from scatter-free projections. The “With Scatter” column shows the strong decomposition bias caused by uncorrected scatter. The “Empirical” correction partially reduced bias but left residual errors (RMSE≈0.076 cm). The “Direct-NN” and “Cross-NN” results both achieved substantially lower errors (RMSE≈0.031 cm), demonstrating that the differentiable Photon Counting CT framework enabled cross-domain training of the scatter model that matched the performance of the directly supervised network. The lower panels of FIG. 16 visualize error maps, confirming suppression of scatter-related streaks and improved uniformity of calcium quantification.

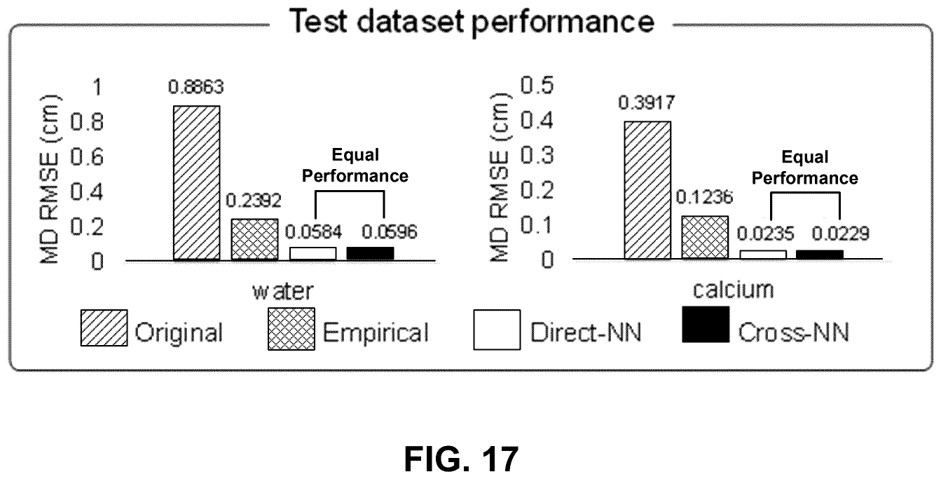

FIG. 17 presents quantitative test-set performance across water and calcium basis images. The mean RMSE of material-decomposition thickness is plotted for the original scatter-contaminated data, the empirically corrected data, the Direct-NN model, and the Cross-NN model. Both Direct-NN and Cross-NN achieved comparable RMSE values (≈0.058 cm for water and ≈0.023 cm for calcium), representing an order-of-magnitude improvement over the empirical method (RMSE≈0.24 cm for water and ≈0.12 cm for calcium) and uncorrected data (RMSE≈0.89 cm for water and ≈0.39 cm for calcium).

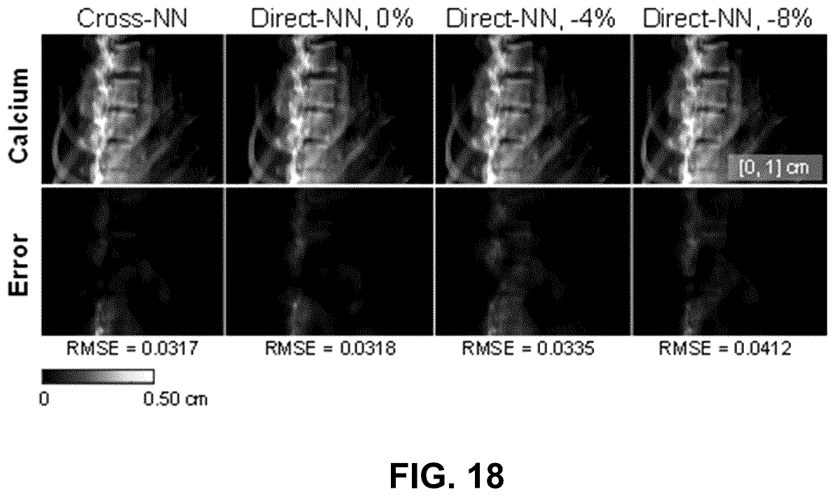

FIG. 18 demonstrates robustness of the proposed cross-domain training under simulation-to-real mismatches. The calcium-basis reconstructions and corresponding error maps are shown for models trained with deliberate spectral-response mismatches in the Direct-NN training (0%, −4%, −8% offsets). The Cross-NN model maintained consistent image fidelity (RMSE≈0.0317 cm) even under substantial mismatch, whereas Direct-NN performance degraded (RMSE increasing from 0.0318 to 0.0412 cm), illustrating that the cross-domain differentiable framework is less sensitive to calibration or modeling errors in the physical measurement domain.

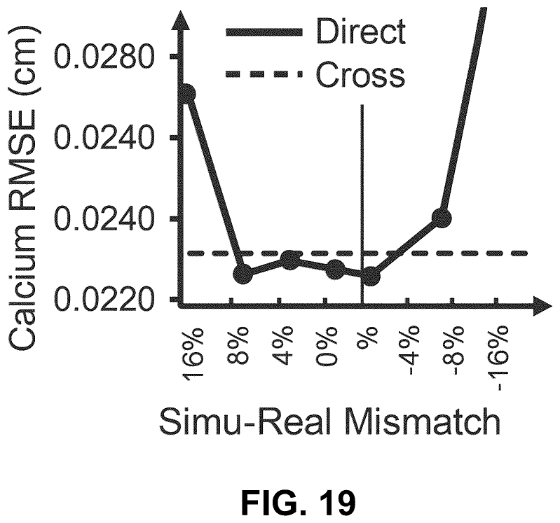

FIG. 19 summarizes the robustness quantitatively, showing the calcium-basis RMSE as a function of simulated mismatch between training and testing conditions. The Cross-NN model (red dashed line) remained stable across all mismatch levels, while the Direct-NN model (blue curve) exhibited pronounced degradation at both high positive and negative mismatches. These results confirm that cross-domain training using the differentiable Photon Counting CT framework provides superior generalization and resilience to physical-model mismatch compared with direct-domain training approaches.

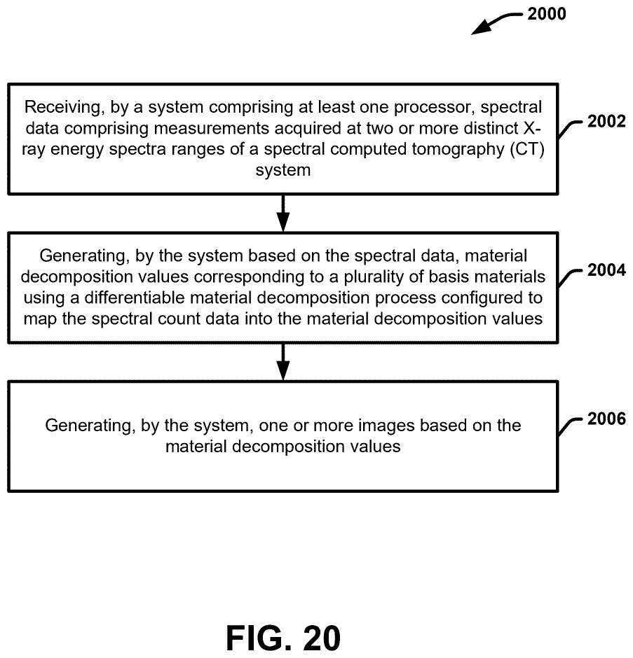

FIG. 20 illustrates an example, computer-implemented method 2000 for generating material-specific images by a spectral CT system, in accordance with one or more embodiments described herein. At 2002, method 2000 comprises receiving, by a system comprising at least one processor (e.g., system 100, system 200, computing device 300, or the like), spectral data (e.g., spectral data 402) comprising measurements acquired at two or more distinct X-ray energy spectra ranges of a spectral CT system. At 2004, method 2000 comprises generating, by the system based on the spectral data, material decomposition values (e.g., MD data 416) corresponding to a plurality of basis materials using a differentiable material decomposition process configured to map the spectral count data into the material decomposition values. At 2006, method 2000 comprises generating, by the system, one or more images (e.g., CT images 422 and/or projection images 424) based on the material decomposition values.

In one implementation of method 2000, the differentiable material decomposition process comprises unrolling, by the system, an iterative maximum-likelihood estimation (MLE) solver into a differentiable computational graph. In another implementation of method 2000, the differentiable material decomposition process comprises determining, by the system, analytical derivatives of an iterative maximum-likelihood estimation (MLE) solver using implicit differentiation.

Still in other embodiments of method 2000, the differentiable material decomposition process comprises executing, by the system (e.g., via execution component 306), using the spectral data as input, a differentiable empirical model (e.g., proxy MD model 606) that generates the material decomposition values as a result of a training process (e.g., training process 500) used to train the differentiable empirical model. In particular, the training process comprises training the differentiable empirical model to minimize a composite loss function comprising an accuracy loss term and a derivative loss term. The accuracy loss term penalizes differences between training material decomposition values output by the differentiable empirical model based on training spectral count data and reference material decomposition values generated by an iterative maximum-likelihood estimation (MLE) solver based on the training input spectral data. The derivative loss term penalizes differences between derivatives of the neural network model and reference derivatives of the MLE solver.

With these embodiments, the derivatives and the reference derivatives respectively represent how the training material decomposition values and the reference material decomposition values change in response to variations in the training spectral data. For example, the variations can comprise noise variations, and wherein the derivative loss term constrains the differentiable empirical model to replicate noise response characteristics of the iterative MLE solver.

In some implementations, method 2000 further comprises performing, by the system, the training process (e.g., training process 500 or the like). To this end, the training process comprises generating, by the system, the reference derivatives using an implicit differentiation process or an unrolled process applied to the iterative MLE solver. The training process further comprises generating, by the system, the reference material decomposition values and the reference derivatives using the iterative MLE solver and based on a forward detector model of a detector of the spectral CT system that generates the spectral data, wherein the forward detector model comprises one or more detector parameters that characterize spectral response properties of the detector.

In accordance with some implementations of method 2000 in which the material decomposition process uses the proxy MD model 606, the duration of time used by the system for executing the differentiable empirical model to generate the material decomposition values is lower than a corresponding duration of time required by the system to execute the iterative MLE solver to produce corresponding material decomposition values based on the spectral count data.

In some implementations, method 2000 further comprises acquiring, by the system, the spectral count data in association with performance a medical image exam on a patient using the spectral CT system. In some embodiments, the spectral CT system corresponds to a PCCT system. In other embodiments, the spectral CT system corresponds to a DECT system.

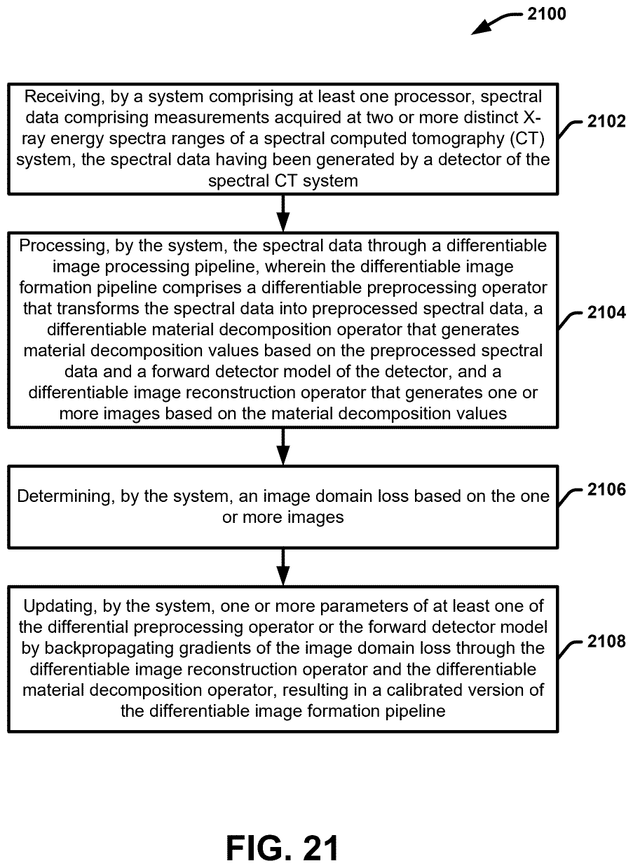

FIG. 21 illustrates an example, computer-implemented method 2100 for cross-domain optimization or calibration of differentiable operators of the disclosed end-to-end differentiable image processing pipeline, in accordance with one or more embodiments. At 2102, method 2100 comprises receiving, by a system comprising at least one processor (e.g., system 100, system 200, computing device 300, or the like), spectral data comprising measurements acquired at two or more distinct X-ray energy spectra ranges of a spectral CT system, the spectral data having been generated by a detector of the spectral CT system (e.g., a PCCT system detector or a DECT system detector) At 2104, method 2100 comprises processing, by the system, the spectral data through a differentiable image processing pipeline (e.g., pipeline 304), wherein the differentiable image formation pipeline comprises a differentiable preprocessing operator that transforms the spectral data into preprocessed spectral data, a differentiable material decomposition operator that generates material decomposition values based on the preprocessed spectral data and a forward detector model of the detector, and a differentiable image reconstruction operator that generates one or more images based on the material decomposition values. At 2106, method 2100 comprises determining an image domain loss based the one or more images. At 2108, method 2100 comprises updating one or more parameters of at least one of the differentiable preprocessing operator or the forward detector model by backpropagating gradients of the image domain loss through the differentiable image reconstruction operator and the differentiable material decomposition operator, resulting in a calibrated version of the differentiable image formation pipeline.

In one or more embodiments, method 2100 further comprises employing, by the system, the calibrated version of the differentiable image processing pipeline to generate subsequent images based on subsequent spectral data acquired by the spectral CT system.

In some implementations, method 2100 further comprise updating one or more additional parameters of the differentiable material decomposition operator based on the backpropagating of the gradients through the differentiable image-reconstruction operator. In some embodiments, method 2100 can include acquiring, by the system, the subsequent spectral data. In other words, the system of method 2100 is or comprises the spectral CT system.

In accordance with method 2100, the forward detector model comprises one or more detector parameters that characterize spectral response properties of the detector, and wherein the updating comprises updating at least one parameter of the one or more detector parameters. For example, the one or more detector parameters can include, but are not limited to, energy bin thresholds, per-pixel gain coefficients, per-channel offset coefficients, detector dead-time coefficients, pile-up coefficients, charge-sharing correction factors, cross-talk correction factors, bin spectral-response functions, quantum-efficiency parameters, detection-probability parameters, spatial-drift correction terms, temporal-drift correction terms, noise-correlation parameters, and dark-count parameters. Based on the computed gradients of the image-domain loss, the system can iteratively adjust these parameters to compensate for drift, miscalibration, or temporal degradation in detector performance. In some embodiments, the updated detector parameters may be written back to detector control registers or configuration files of the spectral CT system to reconfigure the Photon Counting detector in real time or during subsequent acquisitions. Through this process, the spectral CT system achieves adaptive self-calibration, wherein detector hardware and associated models are automatically optimized based on quantitative image-domain feedback.

The differentiable reconstruction operator of method 2100 may be implemented using any of the three methods described herein (e.g., unrolled, implicit differentiation, or proxy MD. For example, in an embodiment the differentiable material decomposition operator comprises a differentiable empirical model (e.g., proxy MD model 606) that generates the material decomposition values as a result of a training process used to train the differentiable empirical model, wherein the training process comprises training the differentiable empirical model to minimize a composite loss function comprising: an accuracy loss term penalizing differences between training material decomposition values output by the differentiable empirical model based on training spectral count data and reference material decomposition values generated by an iterative maximum-likelihood estimation (MLE) solver based on the training input spectral data; and a derivative loss term penalizing differences between derivatives of the neural network model and reference derivatives of the MLE solver.

In various embodiments of method 2100, the differentiable preprocessing operator is selected from the group consisting of: a differentiable spectral response correction operator, a differentiable pile-up correction operator, a differentiable charge sharing correction operator, a differentiable scatter correction operator, and a differentiable noise normalization operator. In some implementations of method 2100, the differentiable preprocessing operator comprises a neural network model (or another trainable type of empirical model, such a regression model, a polynomial, etc.). With these embodiments, the updating may comprise or correspond to training the neural network model.

In some embodiments of method 2100, the spectral CT system comprises a PCCT and wherein the spectral measurements are acquired at two or more distinct X-ray energy spectra ranges respectively corresponding to distinct energy bins of a Photon Counting detector.

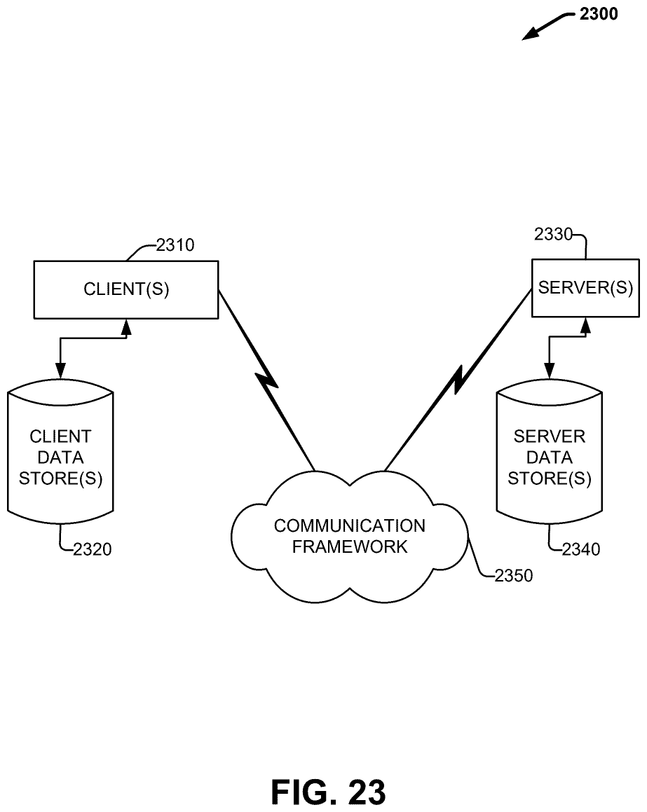

In order to provide a context for the various aspects of the disclosed subject matter, FIGS. 22 and 23 as well as the following discussion are intended to provide a brief, general description of a suitable environment in which the various aspects of the disclosed subject matter may be implemented.

With reference to FIG. 22, a suitable environment 2200 for implementing various aspects of this disclosure includes a computer 2212. The computer 2212 includes a processing unit 2214, a system memory 2216, and a system bus 2218. The system bus 2218 couples system components including, but not limited to, the system memory 2216 to the processing unit 2214. The processing unit 2214 can be any of various available processors. Dual microprocessors and other multiprocessor architectures also can be employed as the processing unit 2214.

The system bus 2218 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 13224), and Small Computer Systems Interface (SCSI).

The system memory 2216 includes volatile memory 2220 and nonvolatile memory 2222. The basic input/output system (BIOS), containing the basic routines to transfer information between elements within the computer 2212, such as during start-up, is stored in nonvolatile memory 2222. By way of illustration, and not limitation, nonvolatile memory 2222 can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory 2220 includes random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 2212 also includes removable/non-removable, volatile/non-volatile computer storage media. FIG. 22 illustrates, for example, a disk storage 2224. Disk storage 2224 includes, but is not limited to, devices like a magnetic disk drive, floppy disk drive, tape drive, Jaz drive, Zip drive, LS drive, flash memory card, or memory stick. The disk storage 2224 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 2224 to the system bus 2218, a removable or non-removable interface is typically used, such as interface 2226.

FIG. 22 also depicts software that acts as an intermediary between users and the basic computer resources described in the suitable operating environment 2200. Such software includes, for example, an operating system 2228. Operating system 2228, which can be stored on disk storage 2224, acts to control and allocate resources of the computer system 2212. System applications 2230 take advantage of the management of resources by operating system 2228 through program modules 2232 and program data 2234, e.g., stored either in system memory 2216 or on disk storage 2224. It is to be appreciated that this disclosure can be implemented with various operating systems or combinations of operating systems.

A user enters commands or information into the computer 2212 through input device(s) 2236. Input devices 2236 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 2214 through the system bus 2218 via interface port(s) 2238. Interface port(s) 2238 include, for example, a serial port, a parallel port, a game port, and a universal serial bus (USB). Output device(s) 2240 use some of the same type of ports as input device(s) 2236. Thus, for example, a USB port may be used to provide input to computer 2212, and to output information from computer 2212 to an output device 2240. Output adapter 2242 is provided to illustrate that there are some output devices 2240 like monitors, speakers, and printers, among other output devices 2240, which require special adapters. The output adapters 2242 include, by way of illustration and not limitation, video and sound cards that provide a means of connection between the output device 2240 and the system bus 2218. It should be noted that other devices and/or systems of devices provide both input and output capabilities such as remote computer(s) 2244.

Computer 2212 can operate in a networked environment using logical connections to one or more remote computers, such as remote computer(s) 2244. The remote computer(s) 2244 can be a personal computer, a server, a router, a network PC, a workstation, a microprocessor based appliance, a peer device or other common network node and the like, and typically includes many or all of the elements described relative to computer 2212. For purposes of brevity, only a memory storage device 2246 is illustrated with remote computer(s) 2244. Remote computer(s) 2244 is logically connected to computer 2212 through a network interface 2248 and then physically connected via communication connection 2250. Network interface 2248 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 2250 refers to the hardware/software employed to connect the network interface 2248 to the bus 2218. While communication connection 2250 is shown for illustrative clarity inside computer 2212, it can also be external to computer 2212. The hardware/software necessary for connection to the network interface 2248 includes, for exemplary purposes only, internal and external technologies such as, modems including regular telephone grade modems, cable modems and DSL modems, ISDN adapters, and Ethernet cards.

FIG. 23 is a schematic block diagram of a sample-computing environment 2300 with which the subject matter of this disclosure can interact. The system 2300 includes one or more client(s) 2310. The client(s) 2310 can be hardware and/or software (e.g., threads, processes, computing devices). The system 2300 also includes one or more server(s) 2330. Thus, system 2300 can correspond to a two-tier client server model or a multi-tier model (e.g., client, middle tier server, data server), amongst other models. The server(s) 2330 can also be hardware and/or software (e.g., threads, processes, computing devices). The servers 2330 can house threads to perform transformations by employing this disclosure, for example. One possible communication between a client 2310 and a server 2330 may be in the form of a data packet transmitted between two or more computer processes.

The system 2300 includes a communication framework 2350 that can be employed to facilitate communications between the client(s) 2310 and the server(s) 2330. The client(s) 2310 are operatively connected to one or more client data store(s) 1020 that can be employed to store information local to the client(s) 2310. Similarly, the server(s) 2330 are operatively connected to one or more server data store(s) 2340 that can be employed to store information local to the servers 2330.

It is to be noted that aspects or features of this disclosure can be exploited in substantially any wireless telecommunication or radio technology, e.g., Wi-Fi; Bluetooth; Worldwide Interoperability for Microwave Access (WiMAX); Enhanced General Packet Radio Service (Enhanced GPRS); Third Generation Partnership Project (3GPP) Long Term Evolution (LTE); Third Generation Partnership Project 2 (3GPP2) Ultra Mobile Broadband (UMB); 3GPP Universal Mobile Telecommunication System (UMTS); High Speed Packet Access (HSPA); High Speed Downlink Packet Access (HSDPA); High Speed Uplink Packet Access (HSUPA); GSM (Global System for Mobile Communications) EDGE (Enhanced Data Rates for GSM Evolution) Radio Access Network (GERAN); UMTS Terrestrial Radio Access Network (UTRAN); LTE Advanced (LTE-A); etc. Additionally, some or all of the aspects described herein can be exploited in legacy telecommunication technologies, e.g., GSM. In addition, mobile as well non-mobile networks (e.g., the Internet, data service network such as internet protocol television (IPTV), etc.) can exploit aspects or features described herein.

While the subject matter has been described above in the general context of computer-executable instructions of a computer program that runs on a computer and/or computers, those skilled in the art will recognize that this disclosure also can or may be implemented in combination with other program modules. Generally, program modules include routines, programs, components, data structures, etc. that perform particular tasks and/or implement particular abstract data types. Moreover, those skilled in the art will appreciate that the inventive methods may be practiced with other computer system configurations, including single-processor or multiprocessor computer systems, mini-computing devices, mainframe computers, as well as personal computers, hand-held computing devices (e.g., PDA, phone), microprocessor-based or programmable consumer or industrial electronics, and the like. The illustrated aspects may also be practiced in distributed computing environments where tasks are performed by remote processing devices that are linked through a communications network. However, some, if not all aspects of this disclosure can be practiced on stand-alone computers. In a distributed computing environment, program modules may be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

In another example, respective components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

Various aspects or features described herein can be implemented as a method, apparatus, system, or article of manufacture using standard programming or engineering techniques. In addition, various aspects or features disclosed in this disclosure can be realized through program modules that implement at least one or more of the methods disclosed herein, the program modules being stored in a memory and executed by at least a processor. Other combinations of hardware and software or hardware and firmware can enable or implement aspects described herein, including a disclosed method(s). The term “article of manufacture” as used herein can encompass a computer program accessible from any computer-readable device, carrier, or storage media. For example, computer readable storage media can include but are not limited to magnetic storage devices (e.g., hard disk, floppy disk, magnetic strips . . . ), optical discs (e.g., compact disc (CD), digital versatile disc (DVD), blu-ray disc (BD) . . . ), smart cards, and flash memory devices (e.g., card, stick, key drive . . . ), or the like.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor may also be implemented as a combination of computing processing units.

In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or methods herein are intended to include, without being limited to including, these and any other suitable types of memory.

It is to be appreciated and understood that components, as described with regard to a particular system or method, can include the same or similar functionality as respective components (e.g., respectively named components or similarly named components) as described with regard to other systems or methods disclosed herein.

What has been described above includes examples of systems and methods that provide advantages of this disclosure. It is, of course, not possible to describe every conceivable combination of components or methods for purposes of describing this disclosure, but one of ordinary skill in the art may recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A method, comprising:
receiving, by a system including at least one processor, spectral data including measurements acquired at two or more distinct X-ray energy spectra ranges of a spectral Computed Tomography (CT) system;
generating, by the system based on the spectral data, material decomposition values corresponding to a plurality of basis materials using a differentiable material decomposition process configured to map the spectral data into the material decomposition values; and
generating, by the system, one or more images based on the material decomposition values.

2. The method of claim 1, wherein the differentiable material decomposition process includes:
unrolling, by the system, an iterative maximum-likelihood estimation (MLE) solver into a differentiable computational graph.

3. The method of claim 1, wherein the differentiable material decomposition process includes:
determining, by the system, analytical derivatives of an iterative maximum-likelihood estimation (MLE) solver using implicit differentiation.

4. The method of claim 1, wherein the differentiable material decomposition process includes:
executing, by the system, using the spectral data as input, a differentiable empirical model that generates the material decomposition values as a result of a training process used to train the differentiable empirical model, wherein the training process includes training the differentiable empirical model to minimize a composite loss function including:
an accuracy loss term penalizing differences between training material decomposition values output by the differentiable empirical model based on training spectral data and reference material decomposition values generated by an iterative maximum-likelihood estimation (MLE) solver based on the training spectral data; and
a derivative loss term penalizing differences between derivatives of the differentiable empirical model and reference derivatives of the iterative MLE solver.

5. The method of claim 4, wherein the derivatives and the reference derivatives respectively represent how the training material decomposition values and the reference material decomposition values change in response to variations in the training spectral data.

6. The method of claim 5, wherein the variations include noise variations, and wherein the derivative loss term constrains the differentiable empirical model to replicate noise response characteristics of the iterative MLE solver.

7. The method of claim 4, further including:
performing, by the system, the training process.

8. The method of claim 4, wherein the training process includes:
generating, by the system, the reference derivatives using an implicit differentiation process or an unrolled process applied to the iterative MLE solver.

9. The method of claim 4, wherein the training process includes:
generating, by the system, the reference material decomposition values and the reference derivatives using the iterative MLE solver and based on a forward detector model of a detector of the spectral CT system that generates the spectral data, wherein the forward detector model includes one or more detector parameters that characterize spectral response properties of the detector.

10. The method of claim 4, wherein a duration of time used by the system for executing the differentiable empirical model to generate the material decomposition values is lower than a corresponding duration of time required by the system to execute the iterative MLE solver to produce corresponding material decomposition values based on the spectral data.

11. The method of claim 1, wherein the spectral CT system includes a photon-counting Computed Tomography system (PCCT), and wherein the measurements are acquired at two or more distinct X-ray energy spectra ranges.

12. A spectral CT system, comprising:
at least one memory storing computer-executable instructions; and
at least one processor configured to execute the computer-executable instructions to perform operations including:
receiving spectral data including measurements acquired at two or more distinct X-ray energy spectra ranges of a spectral Computed Tomography (CT) system, the spectral data having been generated by a detector of the spectral CT system;
processing the spectral data through a differentiable image processing pipeline, wherein the differentiable image processing pipeline includes a differentiable preprocessing operator that transforms the spectral data into preprocessed spectral data, a differentiable material decomposition operator that generates material decomposition values based on the preprocessed spectral data and a forward detector model of the detector, and a differentiable image reconstruction operator that generates one or more images based on the material decomposition values;
determining an image domain loss based on the one or more images; and
updating one or more parameters of at least one of the differentiable preprocessing operator or the forward detector model by backpropagating gradients of the image domain loss through the differentiable image reconstruction operator and the differentiable material decomposition operator, resulting in a calibrated version of the differentiable image processing pipeline.

13. The system of claim 12, wherein the operations further includes:
employing the calibrated version of the differentiable image processing pipeline to generate subsequent images based on subsequent spectral data acquired by the spectral CT system.

14. The system of claim 12, wherein the forward detector model includes one or more detector parameters that characterize spectral response properties of the detector, and wherein the updating includes updating at least one parameter of the one or more detector parameters.

15. The system of claim 14, wherein the one or more detector parameters are selected from the group consisting of: energy bin thresholds, per-pixel gain coefficients, per-channel offset coefficients, detector dead-time coefficients, pile-up coefficients, charge-sharing correction factors, cross-talk correction factors, bin spectral-response functions, quantum-efficiency parameters, detection-probability parameters, spatial-drift correction terms, temporal-drift correction terms, noise-correlation parameters, and dark-count parameters.

16. The system of claim 12, wherein the differentiable material decomposition operator includes a differentiable empirical model that generates the material decomposition values as a result of a training process used to train the differentiable empirical model, wherein the training process includes training the differentiable empirical model to minimize a composite loss function including:
an accuracy loss term penalizing differences between training material decomposition values output by the differentiable empirical model based on training spectral data and reference material decomposition values generated by an iterative maximum-likelihood estimation (MLE) solver based on the training spectral data; and
a derivative loss term penalizing differences between derivatives of the differentiable empirical model and reference derivatives of the iterative MLE solver.

17. The system of claim 12, wherein the differentiable preprocessing operator is selected from the group consisting of: a differentiable spectral response correction operator, a differentiable pile-up correction operator, a differentiable charge sharing correction operator, a differentiable scatter correction operator, and a differentiable noise normalization operator.

18. The system of claim 17, wherein the differentiable preprocessing operator includes a neural network model and wherein the updating includes training the neural network model.

19. The system of claim 12, wherein the spectral CT system includes a photon-counting Computed Tomography system (PCCT), and wherein the measurements are acquired at two or more distinct X-ray energy spectra ranges.

20. A computer program product comprising a non-transitory computer-readable memory having program instructions embodied therewith, the program instructions executable by a processor to cause the processor to:
receive spectral data including measurements acquired at two or more distinct X-ray energy spectra ranges of a spectral Computed Tomography (CT) system;
generate, based on the spectral data, material decomposition values corresponding to a plurality of basis materials using a differentiable material decomposition process configured to map the spectral data into the material decomposition values; and
generate one or more images based on the material decomposition values.
